SEQUENCE LISTING (13 sequences)

SEQ ID NO: 1 - DNA, 2177 bp, Artificial Sequence
  CCNB1 mRNA sequence
cgaacgccttcgcgcgatcgccctggaaacgcattctctgcgaccggcagccgccaatgggaagggagtgagtgccacgaacaggccaataaggagggagcagtgcggggtttaaatctgaggctaggctggctcttctcggcgtgctgcggcggaacggctgttggtttctgctgggtgtaggtccttggctggtcgggcctccggtgttctgcttctccccgctgagctgctgcctggtgaagaggaagccatggcgctccgagtcaccaggaactcgaaaattaatgctgaaaataaggcgaagatcaacatggcaggcgcaaagcgcgttcctacggcccctgctgcaacctccaagcccggactgaggccaagaacagctcttggggacattggtaacaaagtcagtgaacaactgcaggccaaaatgcctatgaagaaggaagcaaaaccttcagctactggaaaagtcattgataaaaaactaccaaaacctcttgaaaaggtacctatgctggtgccagtgccagtgtctgagccagtgccagagccagaacctgagccagaacctgagcctgttaaagaagaaaaactttcgcctgagcctattttggttgatactgcctctccaagcccaatggaaacatctggatgtgcccctgcagaagaagacctgtgtcaggctttctctgatgtaattcttgcagtaaatgatgtggatgcagaagatggagctgatccaaacctttgtagtgaatatgtgaaagatatttatgcttatctgagacaacttgaggaagagcaagcagtcagaccaaaatacctactgggtcgggaagtcactggaaacatgagagccatcctaattgactggctagtacaggttcaaatgaaattcaggttgttgcaggagaccatgtacatgactgtctccattattgatcggttcatgcagaataattgtgtgcccaagaagatgctgcagctggttggtgtcactgccatgtttattgcaagcaaatatgaagaaatgtaccctccagaaattggtgactttgcttttgtgactgacaacacttatactaagcaccaaatcagacagatggaaatgaagattctaagagctttaaactttggtctgggtcggcctctacctttgcacttccttcggagagcatctaagattggagaggttgatgtcgagcaacatactttggccaaatacctgatggaactaactatgttggactatgacatggtgcactttcctccttctcaaattgcagcaggagctttttgcttagcactgaaaattctggataatggtgaatggacaccaactctacaacattacctgtcatatactgaagaatctcttcttccagttatgcagcacctggctaagaatgtagtcatggtaaatcaaggacttacaaagcacatgactgtcaagaacaagtatgccacatcgaagcatgctaagatcagcactctaccacagctgaattctgcactagttcaagatttagccaaggctgtggcaaaggtgtaacttgtaaacttgagttggagtactatatttacaaataaaattggcaccatgtgccatctgtacatattactgttgcatttacttttaataaagcttgtggccccttttacttttttatagcttaactaatttgaatgtggttacttcctactgtagggtagcggaaaagttgtcttaaaaggtatggtggggatatttttaaaaactccttttggtttacctggggatccaattgatgtatatgtttatatactgggttcttgttttatatacctggcttttactttattaatatgagttactgaaggtgatggaggtatttgaaaattttacttccataggacatactgcatgtaagccaagtcatggagaatctgctgcatagctctattttaaagtaaaagtctaccaccgaatccctagtccccctgttttctgtttcttcttgtgattgctgccataattctaagttatttacttttaccactatttaagttatcaactttagctagtatcttcaaactttcactttgaaaaatgagaattttatattctaagccagttttcattttggttttgtgttttggttaataaaacaatactcaaatacaaaaaaaaaaaa

SEQ ID NO: 2 - DNA, 3008 bp, Artificial Sequence
  FANCB transcript variant 1 mRNA sequence
agcgcgctggcgggaggtttggagcagatggataccgtatcgacgtggggcctccggtatgttgccgctgcgttgagtttcataagtacaagccagacctttgatttacctaccctatcttctttactgatgaagctgaaactactttgtgatgcctattgtcccagatctttctgttaattgcctaacaacaaaccagtttagaaacatcaactggaatcaatttgcatttgaaaattagatcagcacaaagacttgatattgtggaatgactagcaaacaagcaatgtcatctaacgaacaagaaaggctcttgtgttataatggggaagtccttgttttccagttgtctaaaggaaattttgcagataaagagcctacaaaaacacccatattacatgtcagaagaatggtatttgacagaggaacaaaagtatttgttcagaagtccactggattttttaccataaaggaagaaaactctcatttaaaaatcatgtgttgcaactgtgtgtcagatttcagaactggaattaacctcccttacattgtgatagaaaaaaataaaaagaataatgtttttgaatattttttactaatccttcacagtactaataaatttgaaatgcgtttgagttttaaactaggctatgagatgaaggatggcctaagggtccttaatggccctttaattttatggaggcatgtcaaagcattcttctttatctcttctcaaactggcaaagttgttagtgtgtcaggtaacttttcctctattcagtgggcaggggagattgaaaatttaggtatggttttattgggactaaaggaatgttgtttatctgaggaagaatgtactcaagagccttcaaaatcagattatgcaatttggaataccaaattttgtgtatattctcttgaaagtcaagaagtattaagtgatatatacattattcctcctgcttacagcagtgtggtgacttatgtacatatttgtgcaactgagatcatcaaaaaccagttaagaatatctctcattgcccttactcgaaagaatcagctgatttcatttcagaatggaactcctaaaaatgtgtgccagcttccatttggagatccttgtgcagttcaacttatggattcaggtggaggaaacctctttttcgttgtatcctttatatccaataatgcttgtgctgtatggaaagagagctttcaggttgctgctaaatgggaaaaacttagcttagtactgatagatgactttattggaagtggaactgaacaagtactcctactttttaaggactccttgaactcagactgcctgacttcatttaaaataacggatcttggaaaaataaactattcgagtgaaccatcagattgcaatgaagatgacttatttgaagacaaacaagagaatcgttacctggtggttccacctctagaaacaggactgaaagtttgtttttcttcttttcgggaattacggcagcatctgttgcttaaggaaaaaattatttcaaaatcttacaaagctttaataaacctagttcaaggaaaagatgataatacgtcaagtgcagaggagaaggaatgtcttgttcctctttgtggtgaagaagaaaattctgtccatatcttagatgaaaagttatcagacaattttcaagattcagaacagctagtagagaagatatggtatcgtgtaatagatgatagcttggttgttggagtgaaaactacatcttctttgaagctgtccctgaatgatgtgactttatcattgttaatggatcaagcccatgactccagatttcggcttctaaagtgtcaaaatagggtgattaagttgagtacaaatcctttcccagcaccatacttgatgccatgtgaaataggattggaagcaaaaagggtcacgttgacccctgatagcaagaaagaggaaagctttgtttgtgaacacccatctaagaaagagtgtgtacagataattactgctgtaacatctctttcaccacttttaacattcagtaaattttgttgcactgtactgctacaaattatggagagagaaagtggtaactgtcctaaagatcgttatgttgtgtgtggcagagtttttttaagtctagaagatctttcaactgggaagtacctactgacatttccaaagaagaaacctatagagcacatggaagatctttttgcacttcttgcagcattccataaatcttgttttcaaatcacatcacccggctatgccctgaattcaatgaaggtgtggctcttagaacatatgaaatgtgaaataatcaaagaatttccagaagtgtacttttgtgaaagaccgggaagtttctatgggacactcttcacttggaaacagagaacaccattcgaagggattttaataatctattccaggaatcaaacagttatgttccagtgccttcataatctcatcagaattctccctataaactgtttcctcaaaaatctaaaatcaggaagtgagaatttcctaattgataatatggcatttactttggagaaggaactagtcacccttagttctctttcttctgccatagctaaacatgaaagcaattttatgcagaggtgtgaagtgagcaaaggaaagagtagtgtcgtcgcggctgctttatcagacagaagggaaaatatccatccctacagaaaagaacttcagagagaaaagaagaaaatgttgcaaacgaacctaaaagtgagtggtgccctttacagagaaataactttgaaagtagctgaggttcagttgaaatcagactttgctgcacagaaactgagtaatttataattataatttcaatttgatcatattttaaaatatgttttcaccaccatataagattttggttctacttgctatatgcctcctttgtaaaaataaacaccgaggcttactgtagtagaaactttagttttgatgatgcacaaaataaacagcagtggttctc

SEQ ID NO: 3 - DNA, 2887 bp, Artificial Sequence
  FANCB transcript variant 2 mRNA sequence
agcgcgctggcgggaggtttggagcagatggataccgtatcgacgtggggcctccggtatgttgccgctgcgttgaggtttagaaacatcaactggaatcaatttgcatttgaaaattagatcagcacaaagacttgatattgtggaatgactagcaaacaagcaatgtcatctaacgaacaagaaaggctcttgtgttataatggggaagtccttgttttccagttgtctaaaggaaattttgcagataaagagcctacaaaaacacccatattacatgtcagaagaatggtatttgacagaggaacaaaagtatttgttcagaagtccactggattttttaccataaaggaagaaaactctcatttaaaaatcatgtgttgcaactgtgtgtcagatttcagaactggaattaacctcccttacattgtgatagaaaaaaataaaaagaataatgtttttgaatattttttactaatccttcacagtactaataaatttgaaatgcgtttgagttttaaactaggctatgagatgaaggatggcctaagggtccttaatggccctttaattttatggaggcatgtcaaagcattcttctttatctcttctcaaactggcaaagttgttagtgtgtcaggtaacttttcctctattcagtgggcaggggagattgaaaatttaggtatggttttattgggactaaaggaatgttgtttatctgaggaagaatgtactcaagagccttcaaaatcagattatgcaatttggaataccaaattttgtgtatattctcttgaaagtcaagaagtattaagtgatatatacattattcctcctgcttacagcagtgtggtgacttatgtacatatttgtgcaactgagatcatcaaaaaccagttaagaatatctctcattgcccttactcgaaagaatcagctgatttcatttcagaatggaactcctaaaaatgtgtgccagcttccatttggagatccttgtgcagttcaacttatggattcaggtggaggaaacctctttttcgttgtatcctttatatccaataatgcttgtgctgtatggaaagagagctttcaggttgctgctaaatgggaaaaacttagcttagtactgatagatgactttattggaagtggaactgaacaagtactcctactttttaaggactccttgaactcagactgcctgacttcatttaaaataacggatcttggaaaaataaactattcgagtgaaccatcagattgcaatgaagatgacttatttgaagacaaacaagagaatcgttacctggtggttccacctctagaaacaggactgaaagtttgtttttcttcttttcgggaattacggcagcatctgttgcttaaggaaaaaattatttcaaaatcttacaaagctttaataaacctagttcaaggaaaagatgataatacgtcaagtgcagaggagaaggaatgtcttgttcctctttgtggtgaagaagaaaattctgtccatatcttagatgaaaagttatcagacaattttcaagattcagaacagctagtagagaagatatggtatcgtgtaatagatgatagcttggttgttggagtgaaaactacatcttctttgaagctgtccctgaatgatgtgactttatcattgttaatggatcaagcccatgactccagatttcggcttctaaagtgtcaaaatagggtgattaagttgagtacaaatcctttcccagcaccatacttgatgccatgtgaaataggattggaagcaaaaagggtcacgttgacccctgatagcaagaaagaggaaagctttgtttgtgaacacccatctaagaaagagtgtgtacagataattactgctgtaacatctctttcaccacttttaacattcagtaaattttgttgcactgtactgctacaaattatggagagagaaagtggtaactgtcctaaagatcgttatgttgtgtgtggcagagtttttttaagtctagaagatctttcaactgggaagtacctactgacatttccaaagaagaaacctatagagcacatggaagatctttttgcacttcttgcagcattccataaatcttgttttcaaatcacatcacccggctatgccctgaattcaatgaaggtgtggctcttagaacatatgaaatgtgaaataatcaaagaatttccagaagtgtacttttgtgaaagaccgggaagtttctatgggacactcttcacttggaaacagagaacaccattcgaagggattttaataatctattccaggaatcaaacagttatgttccagtgccttcataatctcatcagaattctccctataaactgtttcctcaaaaatctaaaatcaggaagtgagaatttcctaattgataatatggcatttactttggagaaggaactagtcacccttagttctctttcttctgccatagctaaacatgaaagcaattttatgcagaggtgtgaagtgagcaaaggaaagagtagtgtcgtcgcggctgctttatcagacagaagggaaaatatccatccctacagaaaagaacttcagagagaaaagaagaaaatgttgcaaacgaacctaaaagtgagtggtgccctttacagagaaataactttgaaagtagctgaggttcagttgaaatcagactttgctgcacagaaactgagtaatttataattataatttcaatttgatcatattttaaaatatgttttcaccaccatataagattttggttctacttgctatatgcctcctttgtaaaaataaacaccgaggcttactgtagtagaaactttagttttgatgatgcacaaaataaacagcagtggttctc

SEQ ID NO: 4 - DNA, 4612 bp, Artificial Sequence
  FANCC transcript variant 1 mRNA sequence
agaatgcactgctgacacgtgtgcgcgcgcgcggctccactgccgggcgaccgcgggaaaattccaaaaaaactcaaaaagccaatacgaggcaaagccaaattttcaagccacagatcccgggcggtggcttcctttccgccactgcccaaactgctgaagcagctcccgcgaggaccacccgatttaatgtgtgccgaccatttccttcagtgctggacaggctgctgtgaagggacatcaccttttcgctttttccaagatggctcaagattcagtagatctttcttgtgattatcagttttggatgcagaagctttctgtatgggatcaggcttccactttggaaacccagcaagacacctgtcttcacgtggctcagttccaggagttcctaaggaagatgtatgaagccttgaaagagatggattctaatacagtcattgaaagattccccacaattggtcaactgttggcaaaagcttgttggaatccttttattttagcatatgatgaaagccaaaaaattctaatatggtgcttatgttgtctaattaacaaagaaccacagaattctggacaatcaaaacttaactcctggatacagggtgtattatctcatatactttcagcactcagatttgataaagaagttgctcttttcactcaaggtcttgggtatgcacctatagattactatcctggtttgcttaaaaatatggttttatcattagcgtctgaactcagagagaatcatcttaatggatttaacactcaaaggcgaatggctcccgagcgagtggcgtccctgtcacgagtttgtgtcccacttattaccctgacagatgttgaccccctggtggaggctctcctcatctgtcatggacgtgaacctcaggaaatcctccagccagagttctttgaggctgtaaacgaggccattttgctgaagaagatttctctccccatgtcagctgtagtctgcctctggcttcggcaccttcccagccttgaaaaagcaatgctgcatctttttgaaaagctaatctccagtgagagaaattgtctgagaaggatcgaatgctttataaaagattcatcgctgcctcaagcagcctgccaccctgccatattccgggttgttgatgagatgttcaggtgtgcactcctggaaaccgatggggccctggaaatcatagccactattcaggtgtttacgcagtgctttgtagaagctctggagaaagcaagcaagcagctgcggtttgcactcaagacctactttccttacacttctccatctcttgccatggtgctgctgcaagaccctcaagatatccctcggggacactggctccagacactgaagcatatttctgaactgctcagagaagcagttgaagaccagactcatgggtcctgcggaggtccctttgagagctggttcctgttcattcacttcggaggatgggctgagatggtggcagagcaattactgatgtcggcagccgaaccccccacggccctgctgtggctcttggccttctactacggcccccgtgatgggaggcagcagagagcacagactatggtccaggtgaaggccgtgctgggccacctcctggcaatgtccagaagcagcagcctctcagcccaggacctgcagacggtagcaggacagggcacagacacagacctcagagctcctgcacaacagctgatcaggcaccttctcctcaacttcctgctctgggctcctggaggccacacgatcgcctgggatgtcatcaccctgatggctcacactgctgagataactcacgagatcattggctttcttgaccagaccttgtacagatggaatcgtcttggcattgaaagccctagatcagaaaaactggcccgagagctccttaaagagctgcgaactcaagtctagaaggcacgcaggccgtgtgggtgcccggcgtgagggatcaggctcgccagggccacaggacaggtgatgacctgtggccacgcatttgtggagtaagtgccctcgctgggctgtgagaatgagctgtacacatcttgggacaatctgctagtatctattttacaaaatgcagagccaggtccctcagcccagactcagtcagacatgttcactaatgactcaagtgagccttcggtactcctggtgcccgcccggccagaccgtcagcttgataattactaaagcaaaggcctgggtgggagaacaggtttctagtttttacccaagtcaagctgcacatctattatttaaaaattcaaagtcttagaaccaagaatttggtcatgaaccattaaagaatttagagagaacttagctctttttagactctttttaggagtcagggatctgggataaagccacactgtcttgctgtatggagaaattcttcaaggggagtcagggtccctcaggcttcccttgtgtctccctggacctgcctgacaggccacaggagcagacagcacacccaagcccgggcctccggcacactctttccactctgtatttgctaaatgatgctaactgctaccaaaaggcccttgggacatcagaggagccggcaggcgaaggtagaggatgtgttccagaaacattagaaggcaggattaattcagttagttagttctcttgttaaatggaaatgggaattggaaattcctgataaagaattggcctggctgggtgcagtggctcacacctgtgatcccagcactttgggaggccaaggcagggggattacttcagcccaggagttccagactgcctggctaacatggcaataccctatctctactaaaaatacaaaaattatcggggtgcaatggcatgcatctgtaatcccagctattcaagaggctgaggcatgaggatctcttgaacccgggaggtgggagttgtagtgagccgagatcatgacactgcactccagcctgggcaacagagcgagaccatctcttaaaaaaaggcattgttagtgtaatctcaaggttaacatttatttcatgtcagtacagggtgctttttcctttcagggacattctggaattgtattggttgtacattcttttgtgtctattctgtttgtcaagtgagtcaagacttgcttttgtccattttgatttgtgtgtattagtctgagtcttggctccgttttgaggtatgagcaaagttttgctggattagaagttaacctttagggaaattccttattttggtatgtggcaatgctaatagatccactgaagatctggaaaattccaggaacttttcacctgagcctttcttctgagaaatgctgcagtcagaagggtgtgctggtaaagtattttggtggcagctgccatcatggtcattgccttcatataacatgcttcgtgctcatggtcattgccttcatataacatgcttcgtgccatcatgatccttgccttcatataacaaacatgcttcgtcagaggtgttggggttgaaaaaggagctgcatgcttcactggagttgagggcctctctcctgttctgactttaagccagaacttgtggctgggccatggaagctgtgactcctctgtggacatggtggcagcagggaacccctagagagaggggccactgggaccaggcctcctgttgtggagggactcctgggacagtcctccaccctgtcctgtggtcctgtgtacagggttggcctcttcctcctcccctgccaggcctctgcccatgccccttccttccttctcctgggactggtgaagctaggcatctggaagacttcttcctagcctggaagccctgacctcggcccatctgcagaatctcccagttccttcacagctgccgagtcctctcacgggtgcggtggaggcggccttgccggtggtgctttctgggcagccaggggttcctgggtgggaggactgtccctctggggacgtggcactgaagtgcctgctggcttcatgtggccctttgccctttcccagcctgagagatgctcaaaggtggggagctgggggagccacccctcggccattccctccacctccaagacaggtggcggccgggcaggcactcttaagcccacctccccctcttgttgccttcgatttcggcaaagcctgggcaggtgccaccgggaaggaatggcatccgagatgctgggcggggacgcggcgtggccgagggggccttgacggcgttggcggggcctgggcacaggggcagccgcagggaggcagggatggcaaggcgtgaagccaccctggaaggaactggaccaaggtcttcagaggtgcgacagggtctggaatctgaccttactctagcaggagtttttgtagactctccctgatagtttagtttttgataaagcatgctggtaaaaccactaccctcagagagagccaaaaatacagaagaggcggagagcgcccctccaaccaggctgttattcccctggactccgtgacatctgtggaattttttagctctttaaaatctgtaatttgttgtctattttttcattctaaataaaacttcagtttgcacctaaaaaaaaaaaaaaaaaaaaaa

SEQ ID NO: 5 - DNA, 4554 bp, Artificial Sequence
  FANCC transcript variant 2 mRNA sequence
gggagccggcgctggggtcgcgcggaagggagcccccggagaggcgggagccgggtgttggcgttttggttctttttgttcattgagcgcaggcagctatgtcttcttcaaaggagaggagcaaagatttaatgtgtgccgaccatttccttcagtgctggacaggctgctgtgaagggacatcaccttttcgctttttccaagatggctcaagattcagtagatctttcttgtgattatcagttttggatgcagaagctttctgtatgggatcaggcttccactttggaaacccagcaagacacctgtcttcacgtggctcagttccaggagttcctaaggaagatgtatgaagccttgaaagagatggattctaatacagtcattgaaagattccccacaattggtcaactgttggcaaaagcttgttggaatccttttattttagcatatgatgaaagccaaaaaattctaatatggtgcttatgttgtctaattaacaaagaaccacagaattctggacaatcaaaacttaactcctggatacagggtgtattatctcatatactttcagcactcagatttgataaagaagttgctcttttcactcaaggtcttgggtatgcacctatagattactatcctggtttgcttaaaaatatggttttatcattagcgtctgaactcagagagaatcatcttaatggatttaacactcaaaggcgaatggctcccgagcgagtggcgtccctgtcacgagtttgtgtcccacttattaccctgacagatgttgaccccctggtggaggctctcctcatctgtcatggacgtgaacctcaggaaatcctccagccagagttctttgaggctgtaaacgaggccattttgctgaagaagatttctctccccatgtcagctgtagtctgcctctggcttcggcaccttcccagccttgaaaaagcaatgctgcatctttttgaaaagctaatctccagtgagagaaattgtctgagaaggatcgaatgctttataaaagattcatcgctgcctcaagcagcctgccaccctgccatattccgggttgttgatgagatgttcaggtgtgcactcctggaaaccgatggggccctggaaatcatagccactattcaggtgtttacgcagtgctttgtagaagctctggagaaagcaagcaagcagctgcggtttgcactcaagacctactttccttacacttctccatctcttgccatggtgctgctgcaagaccctcaagatatccctcggggacactggctccagacactgaagcatatttctgaactgctcagagaagcagttgaagaccagactcatgggtcctgcggaggtccctttgagagctggttcctgttcattcacttcggaggatgggctgagatggtggcagagcaattactgatgtcggcagccgaaccccccacggccctgctgtggctcttggccttctactacggcccccgtgatgggaggcagcagagagcacagactatggtccaggtgaaggccgtgctgggccacctcctggcaatgtccagaagcagcagcctctcagcccaggacctgcagacggtagcaggacagggcacagacacagacctcagagctcctgcacaacagctgatcaggcaccttctcctcaacttcctgctctgggctcctggaggccacacgatcgcctgggatgtcatcaccctgatggctcacactgctgagataactcacgagatcattggctttcttgaccagaccttgtacagatggaatcgtcttggcattgaaagccctagatcagaaaaactggcccgagagctccttaaagagctgcgaactcaagtctagaaggcacgcaggccgtgtgggtgcccggcgtgagggatcaggctcgccagggccacaggacaggtgatgacctgtggccacgcatttgtggagtaagtgccctcgctgggctgtgagaatgagctgtacacatcttgggacaatctgctagtatctattttacaaaatgcagagccaggtccctcagcccagactcagtcagacatgttcactaatgactcaagtgagccttcggtactcctggtgcccgcccggccagaccgtcagcttgataattactaaagcaaaggcctgggtgggagaacaggtttctagtttttacccaagtcaagctgcacatctattatttaaaaattcaaagtcttagaaccaagaatttggtcatgaaccattaaagaatttagagagaacttagctctttttagactctttttaggagtcagggatctgggataaagccacactgtcttgctgtatggagaaattcttcaaggggagtcagggtccctcaggcttcccttgtgtctccctggacctgcctgacaggccacaggagcagacagcacacccaagcccgggcctccggcacactctttccactctgtatttgctaaatgatgctaactgctaccaaaaggcccttgggacatcagaggagccggcaggcgaaggtagaggatgtgttccagaaacattagaaggcaggattaattcagttagttagttctcttgttaaatggaaatgggaattggaaattcctgataaagaattggcctggctgggtgcagtggctcacacctgtgatcccagcactttgggaggccaaggcagggggattacttcagcccaggagttccagactgcctggctaacatggcaataccctatctctactaaaaatacaaaaattatcggggtgcaatggcatgcatctgtaatcccagctattcaagaggctgaggcatgaggatctcttgaacccgggaggtgggagttgtagtgagccgagatcatgacactgcactccagcctgggcaacagagcgagaccatctcttaaaaaaaggcattgttagtgtaatctcaaggttaacatttatttcatgtcagtacagggtgctttttcctttcagggacattctggaattgtattggttgtacattcttttgtgtctattctgtttgtcaagtgagtcaagacttgcttttgtccattttgatttgtgtgtattagtctgagtcttggctccgttttgaggtatgagcaaagttttgctggattagaagttaacctttagggaaattccttattttggtatgtggcaatgctaatagatccactgaagatctggaaaattccaggaacttttcacctgagcctttcttctgagaaatgctgcagtcagaagggtgtgctggtaaagtattttggtggcagctgccatcatggtcattgccttcatataacatgcttcgtgctcatggtcattgccttcatataacatgcttcgtgccatcatgatccttgccttcatataacaaacatgcttcgtcagaggtgttggggttgaaaaaggagctgcatgcttcactggagttgagggcctctctcctgttctgactttaagccagaacttgtggctgggccatggaagctgtgactcctctgtggacatggtggcagcagggaacccctagagagaggggccactgggaccaggcctcctgttgtggagggactcctgggacagtcctccaccctgtcctgtggtcctgtgtacagggttggcctcttcctcctcccctgccaggcctctgcccatgccccttccttccttctcctgggactggtgaagctaggcatctggaagacttcttcctagcctggaagccctgacctcggcccatctgcagaatctcccagttccttcacagctgccgagtcctctcacgggtgcggtggaggcggccttgccggtggtgctttctgggcagccaggggttcctgggtgggaggactgtccctctggggacgtggcactgaagtgcctgctggcttcatgtggccctttgccctttcccagcctgagagatgctcaaaggtggggagctgggggagccacccctcggccattccctccacctccaagacaggtggcggccgggcaggcactcttaagcccacctccccctcttgttgccttcgatttcggcaaagcctgggcaggtgccaccgggaaggaatggcatccgagatgctgggcggggacgcggcgtggccgagggggccttgacggcgttggcggggcctgggcacaggggcagccgcagggaggcagggatggcaaggcgtgaagccaccctggaaggaactggaccaaggtcttcagaggtgcgacagggtctggaatctgaccttactctagcaggagtttttgtagactctccctgatagtttagtttttgataaagcatgctggtaaaaccactaccctcagagagagccaaaaatacagaagaggcggagagcgcccctccaaccaggctgttattcccctggactccgtgacatctgtggaattttttagctctttaaaatctgtaatttgttgtctattttttcattctaaataaaacttcagtttgcacctaaaaaaaaaaaaaaaaaaaaaa

SEQ ID NO: 6 - DNA, 2721 bp, Artificial Sequence
  FANCC transcript variant 3 mRNA sequece
agaatgcactgctgacacgtgtgcgcgcgcgcggctccactgccgggcgaccgcgggaaaattccaaaaaaactcaaaaagccaatacgaggcaaagccaaattttcaagccacagatcccgggcggtggcttcctttccgccactgcccaaactgctgaagcagctcccgcgaggaccacccgatttaatgtgtgccgaccatttccttcagtgctggacaggctgctgtgaagggacatcaccttttcgctttttccaagatggctcaagattcagtagatctttcttgtgattatcagttttggatgcagaagctttctgtatgggatcaggcttccactttggaaacccagcaagacacctgtcttcacgtggctcagttccaggagttcctaaggaagatgtatgaagccttgaaagagatggattctaatacagtcattgaaagattccccacaattggtcaactgttggcaaaagcttgttggaatccttttattttagcatatgatgaaagccaaaaaattctaatatggtgcttatgttgtctaattaacaaagaaccacagaattctggacaatcaaaacttaactcctggatacagggtgtattatctcatatactttcagcactcagatttgataaagaagttgctcttttcactcaaggtcttgggtatgcacctatagattactatcctggtttgcttaaaaatatggttttatcattagcgtctgaactcagagagaatcatcttaatggatttaacactcaaaggcgaatggctcccgagcgagtggcgtccctgtcacgagtttgtgtcccacttattaccctgacagatgttgaccccctggtggaggctctcctcatctgtcatggacgtgaacctcaggaaatcctccagccagagttctttgaggctgtaaacgaggccattttgctgaagaagatttctctccccatgtcagctgtagtctgcctctggcttcggcaccttcccagccttgaaaaagcaatgctgcatctttttgaaaagctaatctccagtgagagaaattgtctgagaaggatcgaatgctttataaaagattcatcgctgcctcaagcagcctgccaccctgccatattccgggttgttgatgagatgttcaggtgtgcactcctggaaaccgatggggccctggaaatcatagccactattcaggtgtttacgcagtgctttgtagaagctctggagaaagcaagcaagcagctgcggtttgcactcaagacctactttccttacacttctccatctcttgccatggtgctgctgcaagaccctcaagatatccctcggggacactggctccagacactgaagcatatttctgaactgctcagagaagcagttgaagaccagactcatgggtcctgcggaggtccctttgagagctggttcctgttcattcacttcggaggatgggctgagatggtggcagagcaattactgatgtcggcagccgaaccccccacggccctgctgtggctcttggccttctactacggcccccgtgatgggaggcagcagagagcacagactatgatggcatatgtcatggcaacctgcaggcatggtgatctccagccttgtggtcagaggcgttctcctgtccccaccgaggtggccagagacgagacgctgccagcccattctcctgcaaaacgaagacccttcttccccaaggtcatttgatctgttattgctcagctgccatatctgccccagggtcgcgtcctccaaggcgtgccctgcagaggcgggctgttctgaggggtgtgccggcagcagagcctggccacagccagggccctgtctcgctccccagcagcccggccagggcttgcttccatcttgactgtctcctttcttggatcagtcaacagatcctcacattactggctttatttacatctcttactaaagtaatcagatgatattaaccactttgcttctgaaaaagaagttggtcatttcctaaataacagagcagctaagtatgggactggataactgtctgtggcaaagacagttgttattttatgtggaatataataattacaggaagcaatgctttgattaaaggcattggcacctcccttgcaagcattgatttgctaggttcttaaaacttcaactgccagtgctctatgctgatcttagtagttaaagaggacaaggctcattttgtatagagccctgcttctcttggctttggcattttctgttgtttgaaaacacacagactaataaagatgtctgcgtattttgtttaaaggcttgcatggcataggaaagaaaaatagttttagattttaaaaagttgaaatttggtgataatttattttggatcagaaataaagctttgtgaaaaaattataacccacgtatgtatttgaggattgctgtctgattaaaaagaaagctttttatttcttcccctttaaaagatcacgttaagctaaggaaaatcctggttgatgtttttaacagtaataagatgtacggggcacatatggtttttaaagcccttttatattctgtcttatttgattctgaataagctaagttggtagatatgtgttcaattccatcacagtcaaaaggttactgacagagaatcacttttgaagtataaagaaataaattattcaataacacttaaaaaaaaaaaa

SEQ ID NO: 7 - DNA, 5204 bp, Artificial Sequence
  FANCD2 transcript variant 1 mRNA sequence
ggcctggcgggaaagtcgaaaactacgggcggcgacggcttctcggaagtaatttaagtgcacaagacattggtcaaaatggtttccaaaagaagactgtcaaaatctgaggataaagagagcctgacagaagatgcctccaaaaccaggaagcaaccactttccaaaaagacaaagaaatctcatattgctaatgaagttgaagaaaatgacagcatctttgtaaagcttcttaagatatcaggaattattcttaaaacgggagagagtcagaatcaactagctgtggatcaaatagctttccaaaagaagctctttcagaccctgaggagacacccttcctatcccaaaataatagaagaatttgttagtggcctggagtcttacattgaggatgaagacagtttcaggaactgccttttgtcttgtgagcgtctgcaggatgaggaagccagtatgggtgcatcttattctaagagtctcatcaaactgcttctggggattgacatactgcagcctgccattatcaaaaccttatttgagaagttgccagaatatttttttgaaaacaagaacagtgatgaaatcaacatacctcgactcattgtcagtcaactaaaatggcttgacagagttgtggatggcaaggacctcaccaccaagatcatgcagctgatcagtattgctccagagaacctgcagcatgacatcatcaccagcctacctgagatcctaggggattcccagcacgctgatgtggggaaagaactcagtgacctactgatagagaatacttcactcactgtcccaatcctggatgtcctttcaagcctccgacttgacccaaacttcctattgaaggttcgccagttggtgatggataagttgtcgtctattagattggaggatttacctgtgataataaagttcattcttcattccgtaacagccatggatacacttgaggtaatttctgagcttcgggagaagttggatctgcagcattgtgttttgccatcacggttacaggcttcccaagtaaagttgaaaagtaaaggacgagcaagttcctcaggaaatcaagaaagcagcggtcagagctgtattattctcctctttgatgtaataaagtcagctattagatatgagaaaaccatttcagaagcctggattaaggcaattgaaaacactgcctcagtatctgaacacaaggtgtttgacctggtgatgcttttcatcatctatagcaccaatactcagacaaagaagtacattgacagggtgctaagaaataagattcgatcaggctgcattcaagaacagctgctccagagtacattctctgttcattacttagttcttaaggatatgtgttcatccattctgtcgctggctcagagtttgcttcactctctagaccagagtataatttcatttggcagtctcctatacaaatatgcatttaagttttttgacacgtactgccagcaggaagtggttggtgccttagtgacccatatctgcagtgggaatgaagctgaagttgatactgccttagatgtccttctagagttggtagtgttaaacccatctgctatgatgatgaatgctgtctttgtaaagggcattttagattatctggataacatatcccctcagcaaatacgaaaactcttctatgttctcagcacactggcatttagcaaacagaatgaagccagcagccacatccaggatgacatgcacttggtgataagaaagcagctctctagcaccgtattcaagtacaagctcattgggattattggtgctgtgaccatggctggcatcatggcggcagacagaagtgaatcacctagtttgacccaagagagagccaacctgagcgatgagcagtgcacacaggtgacctccttgttgcagttggttcattcctgcagtgagcagtctcctcaggcctctgcactttactatgatgaatttgccaacctgatccaacatgaaaagctggatccaaaagccctggaatgggttgggcataccatctgtaatgatttccaggatgccttcgtagtggactcctgtgttgttccggaaggtgactttccatttcctgtgaaagcactgtacggactggaagaatacgacactcaggatgggattgccataaacctcctgccgctgctgttttctcaggactttgcaaaagatgggggtccggtgacctcacaggaatcaggccaaaaattggtgtctccgctgtgcctggctccgtatttccggttactgagactttgtgtggagagacagcataacggaaacttggaggagattgatggtctactagattgtcctatattcctaactgacctggagcctggagagaagttggagtccatgtctgctaaagagcgttcattcatgtgttctctcatatttcttactctcaactggttccgagagattgtaaatgccttctgccaggaaacatcacctgagatgaaggggaaggtgctcactcggttaaagcacattgtagaattgcaaataatcctggaaaagtacttggcagtcaccccagactatgtccctcctcttggaaactttgatgtggaaactttagatataacacctcatactgttactgctatttcagcaaaaatcagaaagaaaggaaaaatagaaaggaaacaaaaaacagatggcagcaagacatcctcctctgacacactttcagaagagaaaaattcagaatgtgaccctacgccatctcatagaggccagctaaacaaggagttcacagggaaggaagaaaagacatcattgttactacataattcccatgcttttttccgagagctggacattgaggtcttctctattctacattgtggacttgtgacgaagttcatcttagatactgaaatgcacactgaagctacagaagttgtgcaacttgggccccctgagctgcttttcttgctggaagatctctcccagaagctggagagtatgctgacacctcctattgccaggagagtcccctttctcaagaacaaaggaagccggaatattggattctcacatctccaacagagatctgcccaagaaattgttcattgtgtttttcaactgctgaccccaatgtgtaaccacctggagaacattcacaactattttcagtgtttagctgctgagaatcacggtgtagttgatggaccaggagtgaaagttcaggagtaccacataatgtcttcctgctatcagaggctgctgcagatttttcatgggctttttgcttggagtggattttctcaacctgaaaatcagaatttactgtattcagccctccatgtccttagtagccgactgaaacagggagaacacagccagcctttggaggaactactcagccagagcgtccattacttgcagaatttccatcaaagcattcccagtttccagtgtgctctttatctcatcagacttttgatggttattttggagaaatcaacagcttctgctcagaacaaagaaaaaattgcttcccttgccagacaattcctctgtcgggtgtggccaagtggggataaagagaagagcaacatctctaatgaccagctccatgctctgctctgtatctacctggagcacacagagagcattctgaaggccatagaggagattgctggtgttggtgtcccagaactgatcaactctcctaaagatgcatcttcctccacattccctacactgaccaggcatacttttgttgttttcttccgtgtgatgatggctgaactagagaagacggtgaaaaaaattgagcctggcacagcagcagactcgcagcagattcatgaagagaaactcctctactggaacatggctgttcgagacttcagtatcctcatcaacttgataaaggtatttgatagtcatcctgttctgcatgtatgtttgaagtatgggcgtctctttgtggaagcatttctgaagcaatgtatgccgctcctagacttcagttttagaaaacaccgggaagatgttctgagcttactggaaaccttccagttggacacaaggctgcttcatcacctgtgtgggcattccaagattcaccaggacacgagactcacccaacatgtgcctctgctcaaaaagaccctggaacttttagtttgcagagtcaaagctatgctcactctcaacaattgtagagaggctttctggctgggcaatctaaaaaaccgggacttgcagggtgaagagattaagtcccaaaattcccaggagagcacagcagatgagagtgaggatgacatgtcatcccaggcctccaagagcaaagccactgaggtatctctacaaaacccaccagagtctggcactgatggttgcattttgttaattgttctaagttggtggagcagaactttgcctacttatgtttattgtcaaatgcttctatgcccatttccattccctccataacagcttctgtgcttatataatttttgggacccagaagaaacaacgacacaatcttagaatcactcctgagtatctcgagttgtggcatttgttatagagttgacaattttctgcattatagcctctcattttccatgaattcatatctgaaaccattttagaagggagaagtcatcgaagtattttctgagtgttgagaagaatgagttaaaccatttaaacacatttgaaacatacaaaaatagaaatgtgaaagcatttggtgaaagccaaagcacagagtcagaagctgccaccttagagaactgaaataaaaatagaagttcttacgcttttttgtggtacagatgctttcgacaatttaaagaaagctaaataaaaatgtagacatggctggcgcagtggctcatgcttgtaatcctagcactttttgaggccaaggtaggaggattgcttgagtccgggagctcaaggcaaagctgcacaacataacaagaccctatctccacaaaaaaaatgaaaaataaacctgggtgcggtggctcacacctgtaatcccagcactttgggaggccgatgtgggcagatcacaaggtcaggagttcaagaccagcctggccaacatagtgaaaccccatctctactgaaaatacaaaaattagctgggtgtggtggcacgtgcctgttatctcagctacttgggaggctga

SEQ ID NO: 8 - DNA, 5134 bp, Artificial Sequence
  FANCD2 transcript variant 2 mRNA sequence
ggcctggcgggaaagtcgaaaactacgggcggcgacggcttctcggaagtaatttaagtgcacaagacattggtcaaaatggtttccaaaagaagactgtcaaaatctgaggataaagagagcctgacagaagatgcctccaaaaccaggaagcaaccactttccaaaaagacaaagaaatctcatattgctaatgaagttgaagaaaatgacagcatctttgtaaagcttcttaagatatcaggaattattcttaaaacgggagagagtcagaatcaactagctgtggatcaaatagctttccaaaagaagctctttcagaccctgaggagacacccttcctatcccaaaataatagaagaatttgttagtggcctggagtcttacattgaggatgaagacagtttcaggaactgccttttgtcttgtgagcgtctgcaggatgaggaagccagtatgggtgcatcttattctaagagtctcatcaaactgcttctggggattgacatactgcagcctgccattatcaaaaccttatttgagaagttgccagaatatttttttgaaaacaagaacagtgatgaaatcaacatacctcgactcattgtcagtcaactaaaatggcttgacagagttgtggatggcaaggacctcaccaccaagatcatgcagctgatcagtattgctccagagaacctgcagcatgacatcatcaccagcctacctgagatcctaggggattcccagcacgctgatgtggggaaagaactcagtgacctactgatagagaatacttcactcactgtcccaatcctggatgtcctttcaagcctccgacttgacccaaacttcctattgaaggttcgccagttggtgatggataagttgtcgtctattagattggaggatttacctgtgataataaagttcattcttcattccgtaacagccatggatacacttgaggtaatttctgagcttcgggagaagttggatctgcagcattgtgttttgccatcacggttacaggcttcccaagtaaagttgaaaagtaaaggacgagcaagttcctcaggaaatcaagaaagcagcggtcagagctgtattattctcctctttgatgtaataaagtcagctattagatatgagaaaaccatttcagaagcctggattaaggcaattgaaaacactgcctcagtatctgaacacaaggtgtttgacctggtgatgcttttcatcatctatagcaccaatactcagacaaagaagtacattgacagggtgctaagaaataagattcgatcaggctgcattcaagaacagctgctccagagtacattctctgttcattacttagttcttaaggatatgtgttcatccattctgtcgctggctcagagtttgcttcactctctagaccagagtataatttcatttggcagtctcctatacaaatatgcatttaagttttttgacacgtactgccagcaggaagtggttggtgccttagtgacccatatctgcagtgggaatgaagctgaagttgatactgccttagatgtccttctagagttggtagtgttaaacccatctgctatgatgatgaatgctgtctttgtaaagggcattttagattatctggataacatatcccctcagcaaatacgaaaactcttctatgttctcagcacactggcatttagcaaacagaatgaagccagcagccacatccaggatgacatgcacttggtgataagaaagcagctctctagcaccgtattcaagtacaagctcattgggattattggtgctgtgaccatggctggcatcatggcggcagacagaagtgaatcacctagtttgacccaagagagagccaacctgagcgatgagcagtgcacacaggtgacctccttgttgcagttggttcattcctgcagtgagcagtctcctcaggcctctgcactttactatgatgaatttgccaacctgatccaacatgaaaagctggatccaaaagccctggaatgggttgggcataccatctgtaatgatttccaggatgccttcgtagtggactcctgtgttgttccggaaggtgactttccatttcctgtgaaagcactgtacggactggaagaatacgacactcaggatgggattgccataaacctcctgccgctgctgttttctcaggactttgcaaaagatgggggtccggtgacctcacaggaatcaggccaaaaattggtgtctccgctgtgcctggctccgtatttccggttactgagactttgtgtggagagacagcataacggaaacttggaggagattgatggtctactagattgtcctatattcctaactgacctggagcctggagagaagttggagtccatgtctgctaaagagcgttcattcatgtgttctctcatatttcttactctcaactggttccgagagattgtaaatgccttctgccaggaaacatcacctgagatgaaggggaaggtgctcactcggttaaagcacattgtagaattgcaaataatcctggaaaagtacttggcagtcaccccagactatgtccctcctcttggaaactttgatgtggaaactttagatataacacctcatactgttactgctatttcagcaaaaatcagaaagaaaggaaaaatagaaaggaaacaaaaaacagatggcagcaagacatcctcctctgacacactttcagaagagaaaaattcagaatgtgaccctacgccatctcatagaggccagctaaacaaggagttcacagggaaggaagaaaagacatcattgttactacataattcccatgcttttttccgagagctggacattgaggtcttctctattctacattgtggacttgtgacgaagttcatcttagatactgaaatgcacactgaagctacagaagttgtgcaacttgggccccctgagctgcttttcttgctggaagatctctcccagaagctggagagtatgctgacacctcctattgccaggagagtcccctttctcaagaacaaaggaagccggaatattggattctcacatctccaacagagatctgcccaagaaattgttcattgtgtttttcaactgctgaccccaatgtgtaaccacctggagaacattcacaactattttcagtgtttagctgctgagaatcacggtgtagttgatggaccaggagtgaaagttcaggagtaccacataatgtcttcctgctatcagaggctgctgcagatttttcatgggctttttgcttggagtggattttctcaacctgaaaatcagaatttactgtattcagccctccatgtccttagtagccgactgaaacagggagaacacagccagcctttggaggaactactcagccagagcgtccattacttgcagaatttccatcaaagcattcccagtttccagtgtgctctttatctcatcagacttttgatggttattttggagaaatcaacagcttctgctcagaacaaagaaaaaattgcttcccttgccagacaattcctctgtcgggtgtggccaagtggggataaagagaagagcaacatctctaatgaccagctccatgctctgctctgtatctacctggagcacacagagagcattctgaaggccatagaggagattgctggtgttggtgtcccagaactgatcaactctcctaaagatgcatcttcctccacattccctacactgaccaggcatacttttgttgttttcttccgtgtgatgatggctgaactagagaagacggtgaaaaaaattgagcctggcacagcagcagactcgcagcagattcatgaagagaaactcctctactggaacatggctgttcgagacttcagtatcctcatcaacttgataaaggtatttgatagtcatcctgttctgcatgtatgtttgaagtatgggcgtctctttgtggaagcatttctgaagcaatgtatgccgctcctagacttcagttttagaaaacaccgggaagatgttctgagcttactggaaaccttccagttggacacaaggctgcttcatcacctgtgtgggcattccaagattcaccaggacacgagactcacccaacatgtgcctctgctcaaaaagaccctggaacttttagtttgcagagtcaaagctatgctcactctcaacaattgtagagaggctttctggctgggcaatctaaaaaaccgggacttgcagggtgaagagattaagtcccaaaattcccaggagagcacagcagatgagagtgaggatgacatgtcatcccaggcctccaagagcaaagccactgaggatggtgaagaagacgaagtaagtgctggagaaaaggagcaagatagtgatgagagttatgatgactctgattagaccccagataaattgttgcctgcttctgtgtctctgccagcctgtgatcattttgtgttagagtttgaaatccgctgtttgcctttcttactggtaggatccttttttgttcctctttttttttttttttttttttttttaaagacggggactcgctgtgtttcccaggctggagtgcagtgctgcaatcttggctcactgcaacctccatctcctaggttcaagcgattctcctgcctcagcctcctgagtagctgggacgacaggcacatgccaccatgcccagctaatttttgtatttttagtagatacggggttttaccatgtcggccagatggtctcaatctcctgaactcatgatccacctgcctcagcctcccaaagtgctgggattacaggcatgagccaccgctcccagccatattttgttcttaaagtggggtctttattaacttgtggacatcatggattgtctaacaccatcacagtccctggctcaggattctaatgtagcattatttattggtttggataaacccagctgtgctacactgcagagtaaaatctctgagtcatgattctggactttgggagctagttttgaaactctgatttattgtagaacttaggcttgtaccaattttacaaataaattctgttctaagttcaaaaaaaaaaaaaaaaaaaaaaaaaaaaaaaa

SEQ ID NO: 9 - DNA, 3665 bp, Artificial Sequence
  FOXM1 transcript variant 1 mRNA sequence
tttcaaacagcggaacaaactgaaagctccggtgccagaccccacccccggccccggcccgggaccccctcccctcccgggatcccccggggttcccaccccgcccgcaccgccggggacccggccggtccggcgcgagcccccgtccggggccctggctcggcccccaggttggaggagcccggagcccgccttcggagctacggcctaacggcggcggcgactgcagtctggagggtccacacttgtgattctcaatggagagtgaaaacgcagattcataatgaaaactagcccccgtcggccactgattctcaaaagacggaggctgccccttcctgttcaaaatgccccaagtgaaacatcagaggaggaacctaagagatcccctgcccaacaggagtctaatcaagcagaggcctccaaggaagtggcagagtccaactcttgcaagtttccagctgggatcaagattattaaccaccccaccatgcccaacacgcaagtagtggccatccccaacaatgctaatattcacagcatcatcacagcactgactgccaagggaaaagagagtggcagtagtgggcccaacaaattcatcctcatcagctgtgggggagccccaactcagcctccaggactccggcctcaaacccaaaccagctatgatgccaaaaggacagaagtgaccctggagaccttgggaccaaaacctgcagctagggatgtgaatcttcctagaccacctggagccctttgcgagcagaaacgggagacctgtgcagatggtgaggcagcaggctgcactatcaacaatagcctatccaacatccagtggcttcgaaagatgagttctgatggactgggctcccgcagcatcaagcaagagatggaggaaaaggagaattgtcacctggagcagcgacaggttaaggttgaggagccttcgagaccatcagcgtcctggcagaactctgtgtctgagcggccaccctactcttacatggccatgatacaattcgccatcaacagcactgagaggaagcgcatgactttgaaagacatctatacgtggattgaggaccactttccctactttaagcacattgccaagccaggctggaagaactccatccgccacaacctttccctgcacgacatgtttgtccgggagacgtctgccaatggcaaggtctccttctggaccattcaccccagtgccaaccgctacttgacattggaccaggtgtttaagccactggacccagggtctccacaattgcccgagcacttggaatcacagcagaaacgaccgaatccagagctccgccggaacatgaccatcaaaaccgaactccccctgggcgcacggcggaagatgaagccactgctaccacgggtcagctcatacctggtacctatccagttcccggtgaaccagtcactggtgttgcagccctcggtgaaggtgccattgcccctggcggcttccctcatgagctcagagcttgcccgccatagcaagcgagtccgcattgcccccaaggtttttggggaacaggtggtgtttggttacatgagtaagttctttagtggcgatctgcgagattttggtacacccatcaccagcttgtttaattttatctttctttgtttatcagtgctgctagctgaggaggggatagctcctctttcttctgcaggaccagggaaagaggagaaactcctgtttggagaagggttttctcctttgcttccagttcagactatcaaggaggaagaaatccagcctggggaggaaatgccacacttagcgagacccatcaaagtggagagccctcccttggaagagtggccctccccggccccatctttcaaagaggaatcatctcactcctgggaggattcgtcccaatctcccaccccaagacccaagaagtcctacagtgggcttaggtccccaacccggtgtgtctcggaaatgcttgtgattcaacacagggagaggagggagaggagccggtctcggaggaaacagcatctactgcctccctgtgtggatgagccggagctgctcttctcagaggggcccagtacttcccgctgggccgcagagctcccgttcccagcagactcctctgaccctgcctcccagctcagctactcccaggaagtgggaggaccttttaagacacccattaaggaaacgctgcccatctcctccaccccgagcaaatctgtcctccccagaacccctgaatcctggaggctcacgcccccagccaaagtagggggactggatttcagcccagtacaaacctcccagggtgcctctgaccccttgcctgaccccctggggctgatggatctcagcaccactcccttgcaaagtgctcccccccttgaatcaccgcaaaggctcctcagttcagaacccttagacctcatctccgtcccctttggcaactcttctccctcagatatagacgtccccaagccaggctccccggagccacaggtttctggccttgcagccaatcgttctctgacagaaggcctggtcctggacacaatgaatgacagcctcagcaagatcctgctggacatcagctttcctggcctggacgaggacccactgggccctgacaacatcaactggtcccagtttattcctgagctacagtagagccctgcccttgcccctgtgctcaagctgtccaccatcccgggcactccaaggctcagtgcaccccaagcctctgagtgaggacagcaggcagggactgttctgctcctcatagctccctgctgcctgattatgcaaaagtagcagtcacaccctagccactgctgggaccttgtgttccccaagagtatctgattcctctgctgtccctgccaggagctgaagggtgggaacaacaaaggcaatggtgaaaagagattaggaaccccccagcctgtttccattctctgcccagcagtctcttaccttccctgatctttgcagggtggtccgtgtaaatagtataaattctccaaattatcctctaattataaatgtaagcttatttccttagatcattatccagagactgccagaaggtgggtaggatgacctggggtttcaattgacttctgttccttgcttttagttttgatagaagggaagacctgcagtgcacggtttcttccaggctgaggtacctggatcttgggttcttcactgcagggacccagacaagtggatctgcttgccagagtcctttttgcccctccctgccacctccccgtgtttccaagtcagctttcctgcaagaagaaatcctggttaaaaaagtcttttgtattgggtcaggagttgaatttggggtgggaggatggatgcaactgaagcagagtgtgggtgcccagatgtgcgctattagatgtttctctgataatgtccccaatcataccagggagactggcattgacgagaactcaggtggaggcttgagaaggccgaaagggcccctgacctgcctggcttccttagcttgcccctcagctttgcaaagagccaccctaggccccagctgaccgcatgggtgtgagccagcttgagaacactaactactcaataaaagcgaaggtggacatgaaaaaaaaaaaaaaaaaaa

SEQ ID NO: 10 - DNA, 3551 bp, Artificial Sequence
  FOXM1 transcript variant 2 mRNA sequence
tttcaaacagcggaacaaactgaaagctccggtgccagaccccacccccggccccggcccgggaccccctcccctcccgggatcccccggggttcccaccccgcccgcaccgccggggacccggccggtccggcgcgagcccccgtccggggccctggctcggcccccaggttggaggagcccggagcccgccttcggagctacggcctaacggcggcggcgactgcagtctggagggtccacacttgtgattctcaatggagagtgaaaacgcagattcataatgaaaactagcccccgtcggccactgattctcaaaagacggaggctgccccttcctgttcaaaatgccccaagtgaaacatcagaggaggaacctaagagatcccctgcccaacaggagtctaatcaagcagaggcctccaaggaagtggcagagtccaactcttgcaagtttccagctgggatcaagattattaaccaccccaccatgcccaacacgcaagtagtggccatccccaacaatgctaatattcacagcatcatcacagcactgactgccaagggaaaagagagtggcagtagtgggcccaacaaattcatcctcatcagctgtgggggagccccaactcagcctccaggactccggcctcaaacccaaaccagctatgatgccaaaaggacagaagtgaccctggagaccttgggaccaaaacctgcagctagggatgtgaatcttcctagaccacctggagccctttgcgagcagaaacgggagacctgtgcagatggtgaggcagcaggctgcactatcaacaatagcctatccaacatccagtggcttcgaaagatgagttctgatggactgggctcccgcagcatcaagcaagagatggaggaaaaggagaattgtcacctggagcagcgacaggttaaggttgaggagccttcgagaccatcagcgtcctggcagaactctgtgtctgagcggccaccctactcttacatggccatgatacaattcgccatcaacagcactgagaggaagcgcatgactttgaaagacatctatacgtggattgaggaccactttccctactttaagcacattgccaagccaggctggaagaactccatccgccacaacctttccctgcacgacatgtttgtccgggagacgtctgccaatggcaaggtctccttctggaccattcaccccagtgccaaccgctacttgacattggaccaggtgtttaagccactggacccagggtctccacaattgcccgagcacttggaatcacagcagaaacgaccgaatccagagctccgccggaacatgaccatcaaaaccgaactccccctgggcgcacggcggaagatgaagccactgctaccacgggtcagctcatacctggtacctatccagttcccggtgaaccagtcactggtgttgcagccctcggtgaaggtgccattgcccctggcggcttccctcatgagctcagagcttgcccgccatagcaagcgagtccgcattgcccccaaggtgctgctagctgaggaggggatagctcctctttcttctgcaggaccagggaaagaggagaaactcctgtttggagaagggttttctcctttgcttccagttcagactatcaaggaggaagaaatccagcctggggaggaaatgccacacttagcgagacccatcaaagtggagagccctcccttggaagagtggccctccccggccccatctttcaaagaggaatcatctcactcctgggaggattcgtcccaatctcccaccccaagacccaagaagtcctacagtgggcttaggtccccaacccggtgtgtctcggaaatgcttgtgattcaacacagggagaggagggagaggagccggtctcggaggaaacagcatctactgcctccctgtgtggatgagccggagctgctcttctcagaggggcccagtacttcccgctgggccgcagagctcccgttcccagcagactcctctgaccctgcctcccagctcagctactcccaggaagtgggaggaccttttaagacacccattaaggaaacgctgcccatctcctccaccccgagcaaatctgtcctccccagaacccctgaatcctggaggctcacgcccccagccaaagtagggggactggatttcagcccagtacaaacctcccagggtgcctctgaccccttgcctgaccccctggggctgatggatctcagcaccactcccttgcaaagtgctcccccccttgaatcaccgcaaaggctcctcagttcagaacccttagacctcatctccgtcccctttggcaactcttctccctcagatatagacgtccccaagccaggctccccggagccacaggtttctggccttgcagccaatcgttctctgacagaaggcctggtcctggacacaatgaatgacagcctcagcaagatcctgctggacatcagctttcctggcctggacgaggacccactgggccctgacaacatcaactggtcccagtttattcctgagctacagtagagccctgcccttgcccctgtgctcaagctgtccaccatcccgggcactccaaggctcagtgcaccccaagcctctgagtgaggacagcaggcagggactgttctgctcctcatagctccctgctgcctgattatgcaaaagtagcagtcacaccctagccactgctgggaccttgtgttccccaagagtatctgattcctctgctgtccctgccaggagctgaagggtgggaacaacaaaggcaatggtgaaaagagattaggaaccccccagcctgtttccattctctgcccagcagtctcttaccttccctgatctttgcagggtggtccgtgtaaatagtataaattctccaaattatcctctaattataaatgtaagcttatttccttagatcattatccagagactgccagaaggtgggtaggatgacctggggtttcaattgacttctgttccttgcttttagttttgatagaagggaagacctgcagtgcacggtttcttccaggctgaggtacctggatcttgggttcttcactgcagggacccagacaagtggatctgcttgccagagtcctttttgcccctccctgccacctccccgtgtttccaagtcagctttcctgcaagaagaaatcctggttaaaaaagtcttttgtattgggtcaggagttgaatttggggtgggaggatggatgcaactgaagcagagtgtgggtgcccagatgtgcgctattagatgtttctctgataatgtccccaatcataccagggagactggcattgacgagaactcaggtggaggcttgagaaggccgaaagggcccctgacctgcctggcttccttagcttgcccctcagctttgcaaagagccaccctaggccccagctgaccgcatgggtgtgagccagcttgagaacactaactactcaataaaagcgaaggtggacatgaaaaaaaaaaaaaaaaaaa

SEQ ID NO: 11 - DNA, 3506 bp, Artificial Sequence
  FOXM1 transcript variant 3 mRNA sequence
tttcaaacagcggaacaaactgaaagctccggtgccagaccccacccccggccccggcccgggaccccctcccctcccgggatcccccggggttcccaccccgcccgcaccgccggggacccggccggtccggcgcgagcccccgtccggggccctggctcggcccccaggttggaggagcccggagcccgccttcggagctacggcctaacggcggcggcgactgcagtctggagggtccacacttgtgattctcaatggagagtgaaaacgcagattcataatgaaaactagcccccgtcggccactgattctcaaaagacggaggctgccccttcctgttcaaaatgccccaagtgaaacatcagaggaggaacctaagagatcccctgcccaacaggagtctaatcaagcagaggcctccaaggaagtggcagagtccaactcttgcaagtttccagctgggatcaagattattaaccaccccaccatgcccaacacgcaagtagtggccatccccaacaatgctaatattcacagcatcatcacagcactgactgccaagggaaaagagagtggcagtagtgggcccaacaaattcatcctcatcagctgtgggggagccccaactcagcctccaggactccggcctcaaacccaaaccagctatgatgccaaaaggacagaagtgaccctggagaccttgggaccaaaacctgcagctagggatgtgaatcttcctagaccacctggagccctttgcgagcagaaacgggagacctgtgcagatggtgaggcagcaggctgcactatcaacaatagcctatccaacatccagtggcttcgaaagatgagttctgatggactgggctcccgcagcatcaagcaagagatggaggaaaaggagaattgtcacctggagcagcgacaggttaaggttgaggagccttcgagaccatcagcgtcctggcagaactctgtgtctgagcggccaccctactcttacatggccatgatacaattcgccatcaacagcactgagaggaagcgcatgactttgaaagacatctatacgtggattgaggaccactttccctactttaagcacattgccaagccaggctggaagaactccatccgccacaacctttccctgcacgacatgtttgtccgggagacgtctgccaatggcaaggtctccttctggaccattcaccccagtgccaaccgctacttgacattggaccaggtgtttaagcagcagaaacgaccgaatccagagctccgccggaacatgaccatcaaaaccgaactccccctgggcgcacggcggaagatgaagccactgctaccacgggtcagctcatacctggtacctatccagttcccggtgaaccagtcactggtgttgcagccctcggtgaaggtgccattgcccctggcggcttccctcatgagctcagagcttgcccgccatagcaagcgagtccgcattgcccccaaggtgctgctagctgaggaggggatagctcctctttcttctgcaggaccagggaaagaggagaaactcctgtttggagaagggttttctcctttgcttccagttcagactatcaaggaggaagaaatccagcctggggaggaaatgccacacttagcgagacccatcaaagtggagagccctcccttggaagagtggccctccccggccccatctttcaaagaggaatcatctcactcctgggaggattcgtcccaatctcccaccccaagacccaagaagtcctacagtgggcttaggtccccaacccggtgtgtctcggaaatgcttgtgattcaacacagggagaggagggagaggagccggtctcggaggaaacagcatctactgcctccctgtgtggatgagccggagctgctcttctcagaggggcccagtacttcccgctgggccgcagagctcccgttcccagcagactcctctgaccctgcctcccagctcagctactcccaggaagtgggaggaccttttaagacacccattaaggaaacgctgcccatctcctccaccccgagcaaatctgtcctccccagaacccctgaatcctggaggctcacgcccccagccaaagtagggggactggatttcagcccagtacaaacctcccagggtgcctctgaccccttgcctgaccccctggggctgatggatctcagcaccactcccttgcaaagtgctcccccccttgaatcaccgcaaaggctcctcagttcagaacccttagacctcatctccgtcccctttggcaactcttctccctcagatatagacgtccccaagccaggctccccggagccacaggtttctggccttgcagccaatcgttctctgacagaaggcctggtcctggacacaatgaatgacagcctcagcaagatcctgctggacatcagctttcctggcctggacgaggacccactgggccctgacaacatcaactggtcccagtttattcctgagctacagtagagccctgcccttgcccctgtgctcaagctgtccaccatcccgggcactccaaggctcagtgcaccccaagcctctgagtgaggacagcaggcagggactgttctgctcctcatagctccctgctgcctgattatgcaaaagtagcagtcacaccctagccactgctgggaccttgtgttccccaagagtatctgattcctctgctgtccctgccaggagctgaagggtgggaacaacaaaggcaatggtgaaaagagattaggaaccccccagcctgtttccattctctgcccagcagtctcttaccttccctgatctttgcagggtggtccgtgtaaatagtataaattctccaaattatcctctaattataaatgtaagcttatttccttagatcattatccagagactgccagaaggtgggtaggatgacctggggtttcaattgacttctgttccttgcttttagttttgatagaagggaagacctgcagtgcacggtttcttccaggctgaggtacctggatcttgggttcttcactgcagggacccagacaagtggatctgcttgccagagtcctttttgcccctccctgccacctccccgtgtttccaagtcagctttcctgcaagaagaaatcctggttaaaaaagtcttttgtattgggtcaggagttgaatttggggtgggaggatggatgcaactgaagcagagtgtgggtgcccagatgtgcgctattagatgtttctctgataatgtccccaatcataccagggagactggcattgacgagaactcaggtggaggcttgagaaggccgaaagggcccctgacctgcctggcttccttagcttgcccctcagctttgcaaagagccaccctaggccccagctgaccgcatgggtgtgagccagcttgagaacactaactactcaataaaagcgaaggtggacatgaaaaaaaaaaaaaaaaaaa

SEQ ID NO: 12 - DNA, 3506 bp, Artificial Sequence
  FOXM1 transcript variant 4 mRNA sequence
tttcaaacagcggaacaaactgaaagctccggtgccagaccccacccccggccccggcccgggaccccctcccctcccgggatcccccggggttcccaccccgcccgcaccgccggggacccggccggtccggcgcgagcccccgtccggggccctggctcggcccccaggttggaggagcccggagcccgccttcggagctacggcctaacggcggcggcgactgcagtctggagggtccacacttgtgattctcaatggagagtgaaaacgcagattcataatgaaaactagcccccgtcggccactgattctcaaaagacggaggctgccccttcctgttcaaaatgccccaagtgaaacatcagaggaggaacctaagagatcccctgcccaacaggagtctaatcaagcagaggcctccaaggaagtggcagagtccaactcttgcaagtttccagctgggatcaagattattaaccaccccaccatgcccaacacgcaagtagtggccatccccaacaatgctaatattcacagcatcatcacagcactgactgccaagggaaaagagagtggcagtagtgggcccaacaaattcatcctcatcagctgtgggggagccccaactcagcctccaggactccggcctcaaacccaaaccagctatgatgccaaaaggacagaagtgaccctggagaccttgggaccaaaacctgcagctagggatgtgaatcttcctagaccacctggagccctttgcgagcagaaacgggagacctgtgatggtgaggcagcaggctgcactatcaacaatagcctatccaacatccagtggcttcgaaagatgagttctgatggactgggctcccgcagcatcaagcaagagatggaggaaaaggagaattgtcacctggagcagcgacaggttaaggttgaggagccttcgagaccatcagcgtcctggcagaactctgtgtctgagcggccaccctactcttacatggccatgatacaattcgccatcaacagcactgagaggaagcgcatgactttgaaagacatctatacgtggattgaggaccactttccctactttaagcacattgccaagccaggctggaagaactccatccgccacaacctttccctgcacgacatgtttgtccgggagacgtctgccaatggcaaggtctccttctggaccattcaccccagtgccaaccgctacttgacattggaccaggtgtttaagcagcagcagaaacgaccgaatccagagctccgccggaacatgaccatcaaaaccgaactccccctgggcgcacggcggaagatgaagccactgctaccacgggtcagctcatacctggtacctatccagttcccggtgaaccagtcactggtgttgcagccctcggtgaaggtgccattgcccctggcggcttccctcatgagctcagagcttgcccgccatagcaagcgagtccgcattgcccccaaggtgctgctagctgaggaggggatagctcctctttcttctgcaggaccagggaaagaggagaaactcctgtttggagaagggttttctcctttgcttccagttcagactatcaaggaggaagaaatccagcctggggaggaaatgccacacttagcgagacccatcaaagtggagagccctcccttggaagagtggccctccccggccccatctttcaaagaggaatcatctcactcctgggaggattcgtcccaatctcccaccccaagacccaagaagtcctacagtgggcttaggtccccaacccggtgtgtctcggaaatgcttgtgattcaacacagggagaggagggagaggagccggtctcggaggaaacagcatctactgcctccctgtgtggatgagccggagctgctcttctcagaggggcccagtacttcccgctgggccgcagagctcccgttcccagcagactcctctgaccctgcctcccagctcagctactcccaggaagtgggaggaccttttaagacacccattaaggaaacgctgcccatctcctccaccccgagcaaatctgtcctccccagaacccctgaatcctggaggctcacgcccccagccaaagtagggggactggatttcagcccagtacaaacctcccagggtgcctctgaccccttgcctgaccccctggggctgatggatctcagcaccactcccttgcaaagtgctcccccccttgaatcaccgcaaaggctcctcagttcagaacccttagacctcatctccgtcccctttggcaactcttctccctcagatatagacgtccccaagccaggctccccggagccacaggtttctggccttgcagccaatcgttctctgacagaaggcctggtcctggacacaatgaatgacagcctcagcaagatcctgctggacatcagctttcctggcctggacgaggacccactgggccctgacaacatcaactggtcccagtttattcctgagctacagtagagccctgcccttgcccctgtgctcaagctgtccaccatcccgggcactccaaggctcagtgcaccccaagcctctgagtgaggacagcaggcagggactgttctgctcctcatagctccctgctgcctgattatgcaaaagtagcagtcacaccctagccactgctgggaccttgtgttccccaagagtatctgattcctctgctgtccctgccaggagctgaagggtgggaacaacaaaggcaatggtgaaaagagattaggaaccccccagcctgtttccattctctgcccagcagtctcttaccttccctgatctttgcagggtggtccgtgtaaatagtataaattctccaaattatcctctaattataaatgtaagcttatttccttagatcattatccagagactgccagaaggtgggtaggatgacctggggtttcaattgacttctgttccttgcttttagttttgatagaagggaagacctgcagtgcacggtttcttccaggctgaggtacctggatcttgggttcttcactgcagggacccagacaagtggatctgcttgccagagtcctttttgcccctccctgccacctccccgtgtttccaagtcagctttcctgcaagaagaaatcctggttaaaaaagtcttttgtattgggtcaggagttgaatttggggtgggaggatggatgcaactgaagcagagtgtgggtgcccagatgtgcgctattagatgtttctctgataatgtccccaatcataccagggagactggcattgacgagaactcaggtggaggcttgagaaggccgaaagggcccctgacctgcctggcttccttagcttgcccctcagctttgcaaagagccaccctaggccccagctgaccgcatgggtgtgagccagcttgagaacactaactactcaataaaagcgaaggtggacatgaaaaaaaaaaaaaaaaaaa

SEQ ID NO: 13 - DNA, 3503 bp, Artificial Sequence
  FOXM1 transcript variant 5 mRNA sequence
tttcaaacagcggaacaaactgaaagctccggtgccagaccccacccccggccccggcccgggaccccctcccctcccgggatcccccggggttcccaccccgcccgcaccgccggggacccggccggtccggcgcgagcccccgtccggggccctggctcggcccccaggttggaggagcccggagcccgccttcggagctacggcctaacggcggcggcgactgcagtctggagggtccacacttgtgattctcaatggagagtgaaaacgcagattcataatgaaaactagcccccgtcggccactgattctcaaaagacggaggctgccccttcctgttcaaaatgccccaagtgaaacatcagaggaggaacctaagagatcccctgcccaacaggagtctaatcaagcagaggcctccaaggaagtggcagagtccaactcttgcaagtttccagctgggatcaagattattaaccaccccaccatgcccaacacgcaagtagtggccatccccaacaatgctaatattcacagcatcatcacagcactgactgccaagggaaaagagagtggcagtagtgggcccaacaaattcatcctcatcagctgtgggggagccccaactcagcctccaggactccggcctcaaacccaaaccagctatgatgccaaaaggacagaagtgaccctggagaccttgggaccaaaacctgcagctagggatgtgaatcttcctagaccacctggagccctttgcgagcagaaacgggagacctgtgatggtgaggcagcaggctgcactatcaacaatagcctatccaacatccagtggcttcgaaagatgagttctgatggactgggctcccgcagcatcaagcaagagatggaggaaaaggagaattgtcacctggagcagcgacaggttaaggttgaggagccttcgagaccatcagcgtcctggcagaactctgtgtctgagcggccaccctactcttacatggccatgatacaattcgccatcaacagcactgagaggaagcgcatgactttgaaagacatctatacgtggattgaggaccactttccctactttaagcacattgccaagccaggctggaagaactccatccgccacaacctttccctgcacgacatgtttgtccgggagacgtctgccaatggcaaggtctccttctggaccattcaccccagtgccaaccgctacttgacattggaccaggtgtttaagcagcagaaacgaccgaatccagagctccgccggaacatgaccatcaaaaccgaactccccctgggcgcacggcggaagatgaagccactgctaccacgggtcagctcatacctggtacctatccagttcccggtgaaccagtcactggtgttgcagccctcggtgaaggtgccattgcccctggcggcttccctcatgagctcagagcttgcccgccatagcaagcgagtccgcattgcccccaaggtgctgctagctgaggaggggatagctcctctttcttctgcaggaccagggaaagaggagaaactcctgtttggagaagggttttctcctttgcttccagttcagactatcaaggaggaagaaatccagcctggggaggaaatgccacacttagcgagacccatcaaagtggagagccctcccttggaagagtggccctccccggccccatctttcaaagaggaatcatctcactcctgggaggattcgtcccaatctcccaccccaagacccaagaagtcctacagtgggcttaggtccccaacccggtgtgtctcggaaatgcttgtgattcaacacagggagaggagggagaggagccggtctcggaggaaacagcatctactgcctccctgtgtggatgagccggagctgctcttctcagaggggcccagtacttcccgctgggccgcagagctcccgttcccagcagactcctctgaccctgcctcccagctcagctactcccaggaagtgggaggaccttttaagacacccattaaggaaacgctgcccatctcctccaccccgagcaaatctgtcctccccagaacccctgaatcctggaggctcacgcccccagccaaagtagggggactggatttcagcccagtacaaacctcccagggtgcctctgaccccttgcctgaccccctggggctgatggatctcagcaccactcccttgcaaagtgctcccccccttgaatcaccgcaaaggctcctcagttcagaacccttagacctcatctccgtcccctttggcaactcttctccctcagatatagacgtccccaagccaggctccccggagccacaggtttctggccttgcagccaatcgttctctgacagaaggcctggtcctggacacaatgaatgacagcctcagcaagatcctgctggacatcagctttcctggcctggacgaggacccactgggccctgacaacatcaactggtcccagtttattcctgagctacagtagagccctgcccttgcccctgtgctcaagctgtccaccatcccgggcactccaaggctcagtgcaccccaagcctctgagtgaggacagcaggcagggactgttctgctcctcatagctccctgctgcctgattatgcaaaagtagcagtcacaccctagccactgctgggaccttgtgttccccaagagtatctgattcctctgctgtccctgccaggagctgaagggtgggaacaacaaaggcaatggtgaaaagagattaggaaccccccagcctgtttccattctctgcccagcagtctcttaccttccctgatctttgcagggtggtccgtgtaaatagtataaattctccaaattatcctctaattataaatgtaagcttatttccttagatcattatccagagactgccagaaggtgggtaggatgacctggggtttcaattgacttctgttccttgcttttagttttgatagaagggaagacctgcagtgcacggtttcttccaggctgaggtacctggatcttgggttcttcactgcagggacccagacaagtggatctgcttgccagagtcctttttgcccctccctgccacctccccgtgtttccaagtcagctttcctgcaagaagaaatcctggttaaaaaagtcttttgtattgggtcaggagttgaatttggggtgggaggatggatgcaactgaagcagagtgtgggtgcccagatgtgcgctattagatgtttctctgataatgtccccaatcataccagggagactggcattgacgagaactcaggtggaggcttgagaaggccgaaagggcccctgacctgcctggcttccttagcttgcccctcagctttgcaaagagccaccctaggccccagctgaccgcatgggtgtgagccagcttgagaacactaactactcaataaaagcgaaggtggacatgaaaaaaaaaaaaaaaaaaa

(19) United States
(12) Patent Application Publication
(10) Pub. No.: US 2015/0275313 A1
(43) Pub. Date: Oct. 1, 2015

(54) MARKER FOR DIAGNOSIS OF BLADDER CANCER RECURRENCE

(71) Applicant: Dong-A University Research Foundation For Industry-Academy Cooperation, Busan (KR)

(72) Inventors: Sun-Hee LEEM, Busan (KR); Yun-Gil Roh, Busan (KR); Tae-Hong Kang, Busan (KR)

(21) Appl. No.: 14/556,074

(22) Filed: Nov. 28, 2014

(30) Foreign Application Priority Data
Mar. 31, 2014 (KR) 10-2014-0037685

(51) Int. Cl. C12Q 1/68

(52) U.S. Cl. CPC C12Q 1/6886; C12Q 2600/158

(57) ABSTRACT

The present disclosure relates to a novel function of a specific gene and has an effect of providing a composition for diagnosis of recurrence of bladder cancer, particularly, non-muscle invasive bladder cancer (NMIBC). Further, according to the present disclosure, it is possible to accurately predict recurrence after surgical tumor removal form a patient with non-muscle invasive bladder cancer, and the present disclosure has an effect of providing information of personalized medicine after surgical tumor removal form a patient with non-muscle invasive bladder cancer.

### CROSS-REFERENCE TO RELATED APPLICATIONS

This application is based on and claims priority from Korean Patent Application No. 10-2014-0037685 filed on Mar. 31, 2014 with the Korean Intellectual Property Office, the disclosure of which is incorporated here in in its entirety by reference.

### TECHNICAL FIELD

The present disclosure relates to a novel biomarker for diagnosis of bladder cancer recurrence and a use thereof, and more particularly, to a use of a marker for diagnosis of recurrence of non-muscle invasive bladder cancer (NMIBC) using expression characteristics of at least one gene selected from the group consisting of CCNB1, FOXM1, FANCB, FANCC, and FANCD2.

Further, the present disclosure relates to a method of predicting a possibility of bladder cancer recurrence after surgical removal of a non-muscle invasive bladder cancer tumor, and a method of providing information of a personalized medicine after surgical removal of a non-muscle invasive bladder cancer tumor.

### BACKGROUND

Bladder cancer is the most frequently occurring cancer of the urinary system. In western countries, the incidence rate for bladder cancer is 16.5 per year per 100,000 population. Meanwhile, in Korea, the incidence rate has been reported as 4.5. As such, the incidence rate of Korea is lower than that of the western countries, but is increasing every year. In Korea, bladder cancer is known as the most frequently occurring cancer of the urinary system (Lee C, et al., 1992).

Bladder cancer is classified into non-muscle invasive bladder cancer and invasive bladder cancer depending on the degree of invasion. The non-muscle invasive bladder cancer is a lesion confined to mucosa without invasion of cancer into muscle and can be relatively simply treated through transurethral resection of bladder tumor or intravesical instillation of anticancer drugs or BCG but has problems of cancer recurrence and progress to invasive cancer. Meanwhile, the invasive bladder cancer refers to a state where cancer invades muscle and needs complicated urinary diversion together with radical cystectomy for treatment and also may lead to a fatal result to a patient.

Therefore, prediction, early detection, and prevention of recurrence and progress after primary treatment are very important.

### SUMMARY

The present disclosure has been made in an effort to provide a composition for marker for diagnosis of bladder cancer recurrence, the composition containing at least one gene selected from the group consisting of CCNB1, FOXM1, FANCB, FANCC, and FANCD2.

Also, the present disclosure has been made in an effort to provide a composition for recurrence marker for diagnosis of bladder cancer, the composition containing a medicine for measuring an expression level of at least one gene selected from the group consisting of CCNB1, FOXM1, FANCB, FANCC, and FANCD2.

Further, the present disclosure has been made in an effort to provide a method of predicting a possibility of bladder cancer recurrence, the method including a step of measuring an expression level of at least one gene selected from the group consisting of CCNB1, FOXM1, FANCB, FANCC, and FANCD2.

Furthermore, the present disclosure has been made in an effort to provide a method of providing information of a personalized medicine after surgical tumor removal, the method including a step of measuring an expression level of at least one gene selected from the group consisting of CCNB1, FOXM1, FANCB, FANCC, and FANCD2. An exemplary embodiment of the present disclosure provides a method of diagnosing a possibility of bladder cancer recurrence, the method including a step of measuring an expression level of at least one gene selected from the group consisting of CCNB1, FOXM1, FANCB, FANCC, and FANCD2.

In the exemplary embodiment of the present disclosure, the method may further include a step of comparing an expression level of at least one gene selected from the group consisting of CCNB1, FOXM1, FANCB, FANCC, and FANCD2 with respect to a specimen obtained from a subject from which non-muscle invasive bladder cancer is removed with an expression level of the gene expressed in a specimen obtained from a normal subject without bladder cancer.

In the exemplary embodiment of the present disclosure, the measurement may be carried out by measuring a level of mRNA of the gene or protein.

In the exemplary embodiment of the present disclosure, when the expression level of at least one gene selected from the group consisting of CCNB1, FOXM1, FANCB, FANCC, and FANCD2 is higher than the expression level expressed in the normal subject, a degree of risk of bladder cancer recurrence may be determined as being high.

Another exemplary embodiment of the present disclosure provides a method of providing information of a personalized medicine after surgical tumor removal, the method including a step of measuring an expression level of at least one gene selected from the group consisting of CCNB1, FOXM1, FANCB, FANCC, and FANCD2.

In the exemplary embodiment of the present disclosure, according to the method of providing information of a personalized medicine after surgical tumor removal, when the expression level of the gene is higher than an expression level of the gene expressed in a specimen obtained from a normal subject without bladder cancer, it is determined to carry out a treatment with Bacillus Calmette-Guerin (BCG) or mytomycin C.

Yet another exemplary embodiment of the present disclosure provides a method of predicting a possibility of bladder cancer recurrence, the method including a step of measuring an expression level of at least one gene selected from the group consisting of CCNB1, FOXM1, FANCB, FANCC, and FANCD2.

In the exemplary embodiment of the present disclosure, the method may further include a step of comparing an expression level of at least one gene selected from the group consisting of CCNB1, FOXM1, FANCB, FANCC, and FANCD2 with respect to a specimen obtained from a subject from which non-muscle invasive bladder cancer is removed with an expression level of the gene expressed in a specimen obtained from a normal subject without bladder cancer.

In the exemplary embodiment of the present disclosure, the measurement may be carried out by measuring a level of mRNA of the gene or protein.

In the exemplary embodiment of the present disclosure, when the expression level of at least one gene selected from the group consisting of CCNB1, FOXM1, FANCB, FANCC, and FANCD2 is higher than the expression level expressed in the normal subject, a degree of risk of bladder cancer recurrence may be determined as being high.

According to the exemplary embodiments of the present disclosure, the present disclosure relates to a novel function of a specific gene and has an effect of providing a composition for diagnosis of recurrence of bladder cancer, particularly, non-muscle invasive bladder cancer (NMIBC). Further, according to the exemplary embodiments of the present disclosure, it is possible to accurately predict recurrence after surgical tumor removal form a patient with non-muscle invasive bladder cancer, and the present disclosure has an effect of providing information of personalized medicine after surgical tumor removal form a patient with non-muscle invasive bladder cancer.

The foregoing summary is illustrative only and is not intended to be in any way limiting. In addition to the illustrative aspects, embodiments, and features described above, further aspects, embodiments, and features will become apparent by reference to the drawings and the following detailed description.

### BRIEF DESCRIPTION OF THE DRAWINGS

FIGS. 1A-1B illustrate recurrence rates of cancer after non-muscle invasive bladder cancer tissues of the Korean cohort are divided into two groups depending on an expression level of CCNB1.

FIGS. 2A-2D illustrate recurrence rates of bladder cancer depending on an expression level of CCNB1 after non-muscle invasive bladder cancer patients are divided into stage Ta patients, stage T1 patients, stage LG (Low Grade) patients, and stage HG (High Grade) patients.

FIGS. 3A-3B illustrate recurrence rates of bladder cancer depending on an expression level of CCNB1 in order to check degrees of recurrence of bladder cancer in patients who are treated with intravesical therapy (IVT) and patient who are not treated with IVT.

FIG. 4 is a schematic diagram illustrating a gene network that interacts with CCNB1.

FIGS. 5A-5E illustrate a result of a check of expression levels of CCNB1, FOXM1, FANCB, FANCC, and FANCD2 genes and bladder cancer recurrence in samples of the Korean cohort.

FIGS. 6A-6F illustrate a result of a check of expression levels of CCNB1, FOXM1, FANCB, FANCC, and FANCD2 genes and bladder cancer recurrence in samples of the Swedish cohort.

FIGS. 7A-7F illustrate a result of a check of expression levels of CCNB1, FOXM1, FANCB, FANCC, and FANCD2 genes and bladder cancer recurrence in samples of the Skane University Hospital patients.

FIGS. 8A-8C illustrate a result of a check of expression levels of FOXM1 and CCNB1 and bladder cancer recurrence in primary tumor tissues and recurrent tumor tissues.

FIGS. 9A-9D illustrates a result of a check of expression levels of FOXM1 and CCNB1 and bladder cancer recurrence after non-muscle invasive cell lines and invasive cell lines are treated with doxorubicin used as an anticancer drug for bladder cancer.

### DETAILED DESCRIPTION

In the following detailed description, reference is made to the accompanying drawing, which forms a part hereof. The illustrative embodiments described in the detailed description, drawing, and claims are not meant to be limiting. Other embodiments may be utilized, and other changes may be made, without departing from the spirit or scope of the subject matter presented here.

Bladder cancer has been known as the most frequently occurring cancer among genito-urinary cancers in Korea, and the fifth most frequently occurring cancer in Korean men among malignant tumors. Bladder cancer is mostly bladder transitional cell carcinoma which can be divided into non-muscle invasive bladder cancer and invasive bladder cancer. The non-muscle invasive bladder cancer accounts for about 70% of the bladder cancer cases. The non-muscle invasive bladder cancer can be treated with preservative treatment such as transurethral resection of bladder tumor or intravesical anticancer therapy and has a high five-year survival rate of about 90%. However, in spite of adequate treatments, it has a high recurrence rate of about 60 to 80%, and 10 to 20% thereof can progress to invasive bladder cancer that invades cystic muscle.

Unlike the non-muscle invasive bladder cancer, the invasive bladder cancer invades blood vessels or lymphatic vessels in early stage and may progress to about 50% or systemic metastasis at the time of diagnosis.

Therefore, in spite of radical cystectomy and adjuvant radiotherapy or anticancer therapy, the invasive bladder cancer has a five-year survival rate of just 20 to 40%. In recent years, in the case of the invasive bladder cancer, bladder preservative treatment has been carried out in some cases to improve the quality of life. However, this treatment also has a risk of recurrence and progress.

Therefore, as for the non-muscle invasive bladder cancer, an early diagnosis and a close follow-up after an adequate treatment are important. Currently, diagnosis and follow-up of the non-muscle invasive bladder cancer rely on cystoscopy and urine cytology typically carried out together. However, cystoscopy is quite painful and uncomfortable for patients and cystoscopy may rarely but possibly cause infection or injury, and, thus, further treatment may be required.

Further, if cancer is very small or is intraepithelial carcinoma, it may be difficult to detect depending on a site of the cancer. Also, urine cytology which is typically carried out together may be different in interpretation among cytopathologists, and it does not have a high sensitivity to bladder cancer with a good differentiation in a low stage.

However, there is no test, which is a non-invasive and capable of diagnosing and accurately predicting recurrence and progress of bladder cancer, as a substitute for cystoscopy and urine cytology.

Therefore, the inventors of the present disclosure have tried to study to discover a novel molecular marker that enables rapid and accurate diagnosis of bladder cancer and/or bladder cancer recurrence.

As a result thereof, the inventors of the present disclosure have found that molecular markers of CCNB1, FOXM1, FANCB, FANCC, and FANCD2 genes can rapidly and accurately detect bladder cancer and/or bladder cancer recurrence.

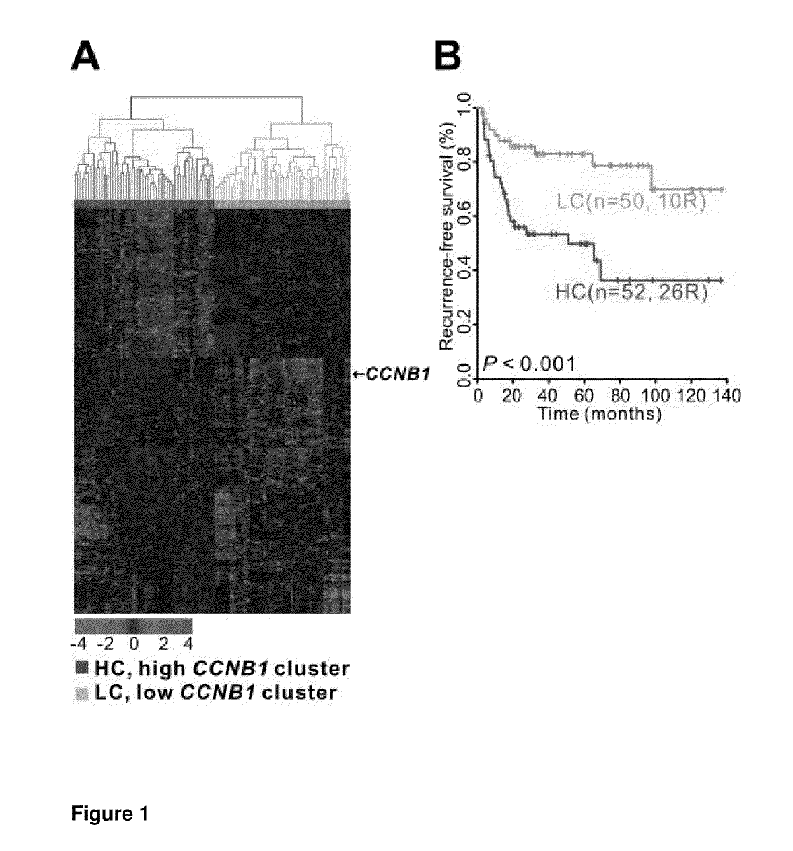

That is, according to an exemplary embodiment of the present disclosure, the inventors of the present disclosure studied a gene expression pattern of cancer using cancer tissues removed from a Korean non-muscle invasive cancer patient by microarray and analyzed progress data of recurrence of each patient. As a result, the inventors of the present disclosure found that gene expression profiles of the tissues removed from the non-muscle invasive cancer patient were divided into two groups (HC: High Expression of CCNB1, LC: Low Expression of CCNB1) depending on an expression level of CCNB1 (refer to FIG. 1). Herein, other 1393 genes illustrating the same expression pattern as CCNB1 were identified. When comparing recurrence rates of cancer, the inventors of the present disclosure found that 10 recurrence cases occurred in the LC group with low expression of the CCNB1 gene, whereas 26 recurrence cases occurred in the HC group with high expression of the CCNB1 gene, and, thus, more recurrence cases of non-muscle invasive cancer occurred in the HC group (refer to FIG. 1).

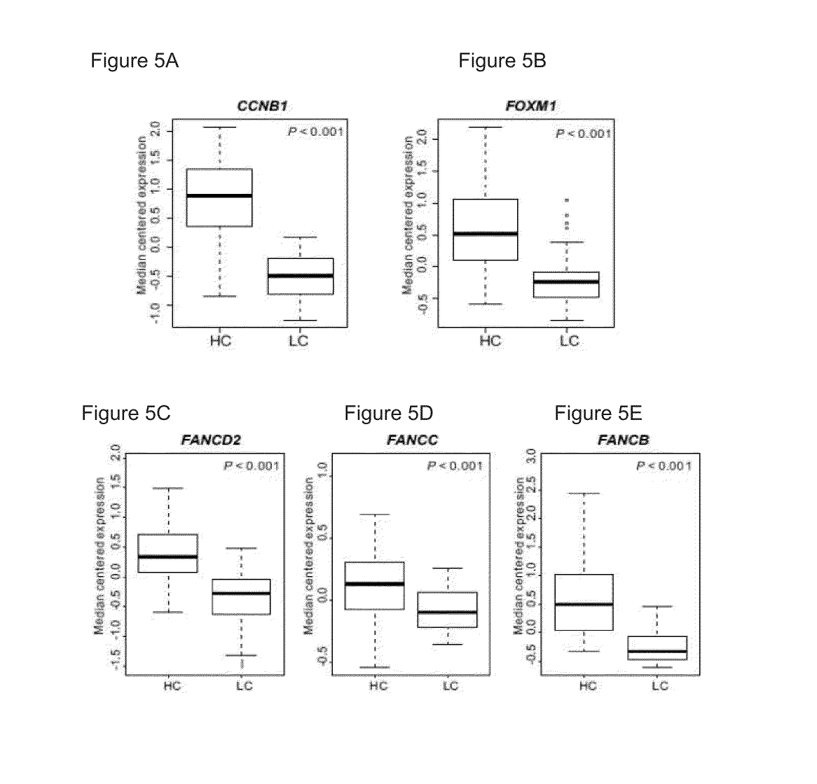

According to another exemplary embodiment, it was found that in the patients with high expression of CCNB1, FOXM1, FANCB, FANCC, and FANCD2 genes relevant to Fanconi anemia pathways together with the CCNB1 gene of the present disclosure, recurrence was significantly increased (refer to FIG. 5).

Therefore, the present disclosure can provide a composition for marker for diagnosis of bladder cancer recurrence, the composition containing at least one gene selected from the group consisting of CCNB1, FOXM1, FANCB, FANCC, and FANCD2.

The term “diagnosis” used in the present disclosure means a check of existence or characteristics of pathological conditions. In view of the objects of the present disclosure, the diagnosis refers to prediction or a check of bladder cancer recurrence after surgery or treatment. Particularly, it is useful for prognosis of non-muscle invasive bladder cancer (NMIBC).

Further, the term “marker for diagnosis” means a substance that enables diagnosis by differentiating bladder cancer cells from normal cells, and includes organic biomolecules such as polypeptides, nucleic acids (for example, mRNA, and the like), lipids, glycolipids, glycoproteins, and saccharides (monosaccharides, disaccharides, oligosaccharides, and the like.) which are increased or decreased in bladder cancer cells rather than normal cells.

The marker for diagnosis of bladder cancer recurrence provided in the present disclosure is a gene or a protein to be discriminately expressed in bladder cancer cells rather than normal cells and may be at least one gene or protein selected from the group consisting of CCNB1, FOXM1, FANCB, FANCC, and FANCD2. Preferably, the CCNB1 may have a base sequence described as SEQ. ID. No. 1, the FANCB may have a base sequence described as SEQ. ID. No. 2 or 3, the FANCC may have a base sequence described as SEQ. ID. No. 4, 5, or 6, FANCD2 may have a base sequence described as SEQ. ID. No. 7 or 8, and the FOXM1 may have a base sequence described as SEQ. ID. No. 9 to 13.

In the present disclosure, a primer or a probe specific to the CCNB1, FOXM1, FANCB, FANCC, and FANCD2 genes may be a primer or a probe which can specifically amplify the whole or specific region of the CCNB1, FOXM1, FANCB, FANCC, and FANCD2 genes, and the primer or the probe can be designed by a method known in the art.

In the present disclosure, the term “primer” refers to a single-stranded oligonucleotide capable of initiating a template-directed DNA synthesis in an appropriate buffer under an appropriate condition (that is, in the presence of four different nucleoside triphosphates and polymerase) at a proper temperature. The appropriate length of the primer may vary according to various factors, for example, temperature and the purpose of use. Further, the primer sequence does not necessarily need to be completely complementary to a part of the sequence of the template, but just needs to be sufficiently complementary to be hybridized with the template and perform the unique function of the primers. Accordingly, the primer of the present disclosure does not necessarily need to be completely complementary to the nucleotide sequence of the CCNB1, FOXM1, FANCB, FANCC, and FANCD2 genes serving as the template, but just needs to be sufficiently complementary to be hybridized with this gene sequence and perform the primer function. Preferably, the primer according to the present disclosure is used for gene amplification.

The term “amplification” or “amplifying” refers to a reaction in which nucleic acid molecules are amplified. Such gene amplifications are well known in the art. Examples are polymerase chain reaction (PCR), reverse-transcription polymerase chain reaction (RT-PCR), ligase chain reaction (LCR), transcription mediated amplification (TMA), nucleic acid based sequence amplification (NASBA), and the like.

In the present disclosure, the term “probe” refers to a linear oligomer of natural or modified monomers or linkages, including deoxyribonucleotides and ribonucleotides, which is capable of specifically hybridized with a target nucleotide sequence and occurring naturally or synthesized artificially.

The probe according to the present disclosure may be a single chain, and preferably, the probe may be an oligodeoxyribonucleotide. The probe of the present disclosure may include naturally occurring dNMP (that is, dAMP, dGMP, dCMP, and dTMP), nucleotide analogs, or nucleotide derivatives. Further, the probe of the present disclosure can also include ribonucleotides. For example, the probe of the present disclosure may include nucleotides with backbone modifications such as peptide nucleic acid (PNA) (M. Egholm et al., Nature, 365:566-568 (1993)), phosphorothioate DNA, phosphorodithioate DNA, phosphoramidate DNA, amide-linked DNA, MMI-linked DNA, 2′-O-methyl RNA, alpha-DNA and methylphosphonate DNA, nucleotides with sugar modifications such as 2′-O-methyl RNA, 2′-fluoro RNA, 2′-amino RNA, 2′-O-alkyl DNA, 2′-O-allyl DNA, 2′-O-alkynyl DNA, hexose DNA, pyranosyl RNA, and anhydrohexitol DNA, and nucleotides having base modifications such as C-5 substituted pyrimidines (substituents including fluoro-, bromo-, chloro-, iodo-, methyl-, ethyl-, vinyl-, formyl-, ethynyl-, propynyl-, alkynyl-, thiazolyl-, imidazolyl-, pyridyl-), 7-deazapurines with C-7 substituents (substituents including fluoro-, bromo-, chloro-, iodo-, methyl-, ethyl-, vinyl-, formyl-, alkynyl-, alkenyl-, thiazolyl-, imidazolyl-, pyridyl-), inosine, and diaminopurine.

Furthermore, in the present disclosure, the substance for measuring the protein level may include polyclonal antibodies, monoclonal antibodies, and recombinant antibodies which specifically bind to the CCNB1, FOXM1, FANCB, FANCC, and FANCD2 protein.

In the present disclosure, since the CCNB1, FOXM1, FANCB, FANCC, and FANCD2 protein were identified as marker proteins for diagnosing bladder cancer recurrence as described above, a method of producing an antibody using the above-described proteins can be easily carried out by techniques generally known to those skilled in the art. The production of, for example, polyclonal antibodies may be carried out using a method widely known in the art, which includes injecting the CCNB1, FOXM1, FANCB, FANCC, and FANCD2 antigen into an animal and collecting blood samples from the animal to obtain sera containing antibodies. Such polyclonal antibodies may be prepared from a certain animal host, such as goats, rabbits, sheep, monkeys, horses, pigs, cows, and dogs. Monoclonal antibodies may be prepared by a method widely known in the art, such as a hybridoma method (Kohler et al., European Journal of Immunology 6: 511-519, 1976), or a phage antibody library technique (Clackson et al., Nature, 352: 624-628, 1991; Marks et al., J. Mol. Biol., 222: 58, 1-597, 1991).

The antibodies of the present disclosure include complete forms, each of which consists of two full-length light chains and two full-length heavy chains, as well as functional fragments of antibody molecules. The functional fragments of antibody molecules refer to fragments retaining at least an antigen-binding function, and include Fab, F(ab′), F(ab′)2, Fv, and the like.

Further, the present disclosure may provide a kit for diagnosis of bladder cancer recurrence, the kit containing the composition for diagnosis of bladder cancer recurrence according to the present disclosure.

The composition for diagnosis of bladder cancer recurrence contained in the kit for diagnosis of bladder cancer recurrence of the present disclosure may contain a substance for measuring a level of mRNA of at least one gene selected from the group consisting of CCNB1, FOXM1, FANCB, FANCC, and FANCD2 or protein as described above. A substance for measuring the mRNA of the gene may include a primer or a probe, and a substance for measuring the protein may include an antibody, and the definitions thereof are the same as described above.

If the kit for diagnosis of bladder cancer recurrence of the present disclosure is used in a PCR amplification procedure, the kit of the present disclosure may selectively include reagents required for PCR amplification, for example, buffer, DNA polymerase (for example, thermostable DNA polymerase obtained from Thermus aquaticus (Taq), Thermus thermophilus (Tth), Thermus filiformis, Thermis flavus, Thermococcus literalis, or Pyrococcus furiosus (Pfu)), DNA co-polymerase, and dNTPs. If the kit for diagnosis of bladder cancer recurrence of the present disclosure is applied to immunoassay, the kit of the present disclosure may selectively comprise a secondary antibody and a labeled substrate.

Further, the kit for diagnosis of bladder cancer recurrence of the present disclosure may be made of a plurality of packagings or compartments including the above-described reagent components. A kind of the kit which can be manufactured in the present disclosure is not limited thereto, and the kit may be a RT-PCR kit, a DNA chip kit, or a protein chip kit.

Further, the present disclosure may provide a microarray for diagnosis of bladder cancer recurrence, the microarray containing the composition for diagnosis of bladder cancer recurrence according to the present disclosure.

In the microarray of the present disclosure, the primer, probe, and antibody for measuring the expression level of the CCNB1, FOXM1, FANCB, FANCC, and FANCD2 protein, or the gene encoding the same serve as hybridizable array elements and are immobilized on substrates. A preferable substrate includes suitable solid or semi-solid supporters, such as membrane, filter, chip, slide, wafer, fiber, magnetic or nonmagnetic bead, gel, tubing, plate, polymer, microparticle, and capillary tube. The hybridizable array elements are arranged and immobilized on the substrate. Such immobilization occurs through chemical binding or covalent binding such as UV. For example, the hybridizable array elements are bound to a glass surface modified to contain an epoxy compound or an aldehyde group or to a polylysine-coated surface by UV irradiation. Further, the hybridizable array elements may be bound to a substrate through linkers (for example, ethylene glycol oligomer and diamine).

If samples to be applied to the microarray of the present disclosure are nucleic acids, they may be labeled, and hybridized with array elements on microarray. Various hybridization conditions are applicable, and for the detection and analysis of the extent of hybridization, various methods are available depending on labels used.

Further, the present disclosure may provide a method of predicting and diagnosing bladder cancer recurrence by measuring an expression level of a marker gene for diagnosis of bladder cancer recurrence. Preferably, the method may include (a) a step of measuring an expression level of at least one gene selected from the group consisting of CCNB1, FOXM1, FANCB, FANCC, and FANCD2 present in biological samples or an expression level of a protein; and (b) a step of comparing the measurement result of the step (a) with the expression level of the gene in samples from the normal control group or the expression level of the protein.

The above-described method for measuring the level of at least one gene selected from the group consisting of CCNB1, FOXM1, FANCB, FANCC, and FANCD2 or the level of protein may be carried out by a method including a known process for isolating mRNA or protein from a biological sample using the known art.

In the present disclosure, the term “biological sample” refers to samples derived from living organisms which show a difference in the expression level of at least one gene selected from the group consisting of CCNB1, FOXM1, FANCB, FANCC, and FANCD2 or the protein level from a normal control group depending on the degree of onset or progress of bladder cancer, and the samples, for example, may include tissues, cells, blood, serum, plasma, saliva, urine, and the like, but are not limited thereto.

Preferably, the measurement of the expression level of at least one gene selected from the group consisting of CCNB1, FOXM1, FANCB, FANCC, and FANCD2 involves measuring mRNA levels. Methods for measuring mRNA levels include reverse transcriptase-polymerase chain reaction (RT-PCR), real time reverse transcriptase-polymerase chain reaction, RNase protection analysis, Northern blot, DNA chip, and the like, but are not limited thereto.

The measurement of the level of at least one protein selected from the group consisting of CCNB1, FOXM1, FANCB, FANCC, and FANCD2 can be carried out using an antibody. In this case, the CCNB1, FOXM1, FANCB, FANCC, and FANCD2 proteins in a biological sample and an antibody specific thereto form binding products, that is, antigen-antibody complexes. An amount of the formed antigen-antibody complexes may be quantitatively determined by measuring a signal size of a detection label. Such a detection label may be selected from the group consisting of enzymes, fluorescent substances, ligands, luminescent substances, microparticles, redox molecules, and radioactive isotopes, but the present disclosure is not limited thereto. Analysis methods for measuring protein levels include, but are not limited to, Western blotting, ELISA, radioimmunodiffusion, ouchterlony immunodiffusion, rocket immunoelectrophoresis, immunohistochemistry, immunoprecipitation assay, complement fixation assay, FACS, protein chip assay, and the like.

Accordingly, with the detection methods of the present disclosure, it is possible to determine the expression level of mRNA or protein of at least one gene selected from the group consisting of CCNB1, FOXM1, FANCB, FANCC, and FANCD2 in a normal control group samples and the expression level of mRNA or protein of at least one gene selected from the group consisting of CCNB1, FOXM1, FANCB, FANCC, and FANCD2 in patients who underwent a medicine treatment for non-invasive bladder cancer or surgical tumor removal. Bladder cancer recurrence can be predicted and diagnosed by comparing the expression level with that of the control group.

In the present disclosure, when an expression level of the CCNB1, FOXM1, FANCB, FANCC, and FANCD2 gene or an expression level of protein in the patients who underwent a medicine treatment for non-invasive bladder cancer or surgical tumor removal is increased as compared with the normal control group, it can be predicted or diagnosed that non-invasive bladder cancer is highly likely to recur.

Accordingly, among the patients who underwent a medicine treatment for non-invasive bladder cancer or surgical tumor removal, a patient group predicted or diagnosed as a high possibility of non-invasive bladder cancer recurrence can be compared with a patient group with a low possibility of non-invasive bladder cancer recurrence, and, thus, it is possible to prescribe a personalized medicine suitable therefor.

Hereinafter, the present disclosure will be described in further detail with reference to the following examples. It will be obvious to a person having ordinary skill in the art that these examples are merely for illustrative purposes, and the scope of the present disclosure should not be construed as being limited to these examples.

### Example 1

### Preparation of Experiment

The samples used in the present disclosure were collected by Department of Urology, Chungbuk National University College of Medicine and divided by the frozen tissue bank for experiment. Further, clinical information of the European patient group, the SSH cohort (Swedish patient group), and the SUH cohort (Skane University Hospital patient group) for testing the present disclosure were as listed in the following Table 1.

[Table 1] Clinical information of Korean cohort used in the present disclosure and clinical information of European cohort, SSH cohort (Swedish patient group), and SUH cohort (Skane University Hospital patient group) used for testing the present disclosure

### Example 2

### Study on Gene Expression Pattern of Cancer Using Non-Muscle Invasive Cancer

The inventors of the present disclosure studied a gene expression pattern of cancer using a cancer tissue removed from a Korean non-muscle invasive cancer patient by microarray and analyzed progress data of recurrence of each patient.

Firstly, a cluster analysis of bladder cancer patients were carried out using expression values of 1,393 genes showing the same expression pattern as a CCNB1 gene, and the bladder cancer patients were divided into two groups depending on an expression cluster pattern of the genes (refer to FIG. 1). In the patient groups divided into two groups, the patient groups were respectively designated as “HC” and “LC” (HC: High Expression of CCNB1, LC: Low Expression of CCNB1) depending on a high or low expression value of the CCNB1 gene in the patient groups divided into the two groups, and recurrence rates of the patients were measured.

As a result, in terms of cancer recurrence rate, it was found that in the LC group with low expression of the CCNB1 gene, 10 recurrence cases occurred, whereas in the HC group with high expression of the CCNB1 gene, 26 recurrence cases occurred, and, thus, more recurrence cases of non-muscle invasive cancer occurred in the HC group.

### Example 3

### Study on Gene Expression Pattern of Cancer Group Classified by Clinical Pathological Method

“The stage Ta” refers to a stage of the lowest grade bladder cancer according to the TNM classification that classifies bladder cancer, of which invasion is confined to the most superficial layer of bladder, and the state “the stage T1” refers to a stage of bladder cancer, of which invasion progresses to the subepithelial connective tissue of bladder. Both of the two stages refer to stages of non-muscle invasive bladder cancer without invasion of tumor cells into muscle of bladder.

Meanwhile, a grade of bladder cancer refers to a histological classification system depending on a differentiation level of cancer cell and is different from a stage. According to WHO 2003 standard, in the case of low grade, there is no change or a slight increase in cell density and a cell nucleus has a uniform circular shape or shows slight polymorphism; no nucleolus is present or most nucleoli are not distinct; and cellular mitosis is rarely observed. Meanwhile, in the case of high grade, intercellular polarity disappears and intercellular adhesion is decreased; a cell nucleus shows severe polymorphism in shape; several nucleoli are distinctly observed; and cellular mitosis is also often observed. It is known that if a bladder cancer cell is less differentiated (low grade), prognosis of a patient is relatively good, and if a bladder cancer cell is more differentiated (high grade), prognosis is severely deteriorated. Even if a stage is low, a grade can be often high, or vice versa. Therefore, it is necessary to classify cases for treatment.

The inventors of the present disclosure found that whether non-muscle invasive cancer recur or not can be determined by measuring an expression level of CCNB1 according to an experiment result of Example 2, and checked whether such a result can be obtained from the stage Ta, the stage T1, the LG (Low Grade) group, and the HG (High Grade) group classified by a clinical pathological method.

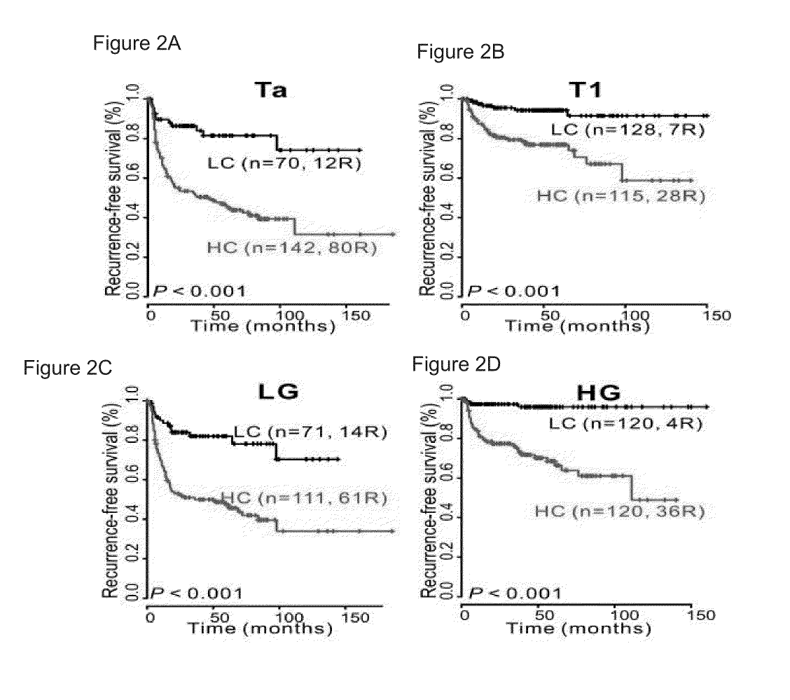

The stage Ta, the stage T1, the LG (Low Grade) group, and the HG (High Grade) group were divided into two groups depending on an expression level of CCNB1, and it was found that with respect to all of these four groups, more recurrence cases of non-invasive bladder cancer occurred in the HC group with a high expression level of CCNB1 rather than the LC group with a low expression level of CCNB1 (refer to FIG. 2).

Further, the inventors of the present disclosure studied relevance between the clinical information of the Korean patient group or an expression level of CCNB1 as found from and the results of Examples 1 and 2 and recurrence.

As a result thereof, it was found that relevance between the clinical information (sex, age, stage, tumor size, presence or absence of treatment, and the like.) and recurrence did not show any significance, but only when an expression level of CCNB1 used in the present disclosure was high (HC), there is a significant relevance to a risk of recurrence (refer to Table 2).

TABLE 2
Result of multivariate Cox regression analysis predicting cancer
recurrence in Korean cohort

| | Recurrence | |
|---|---|---|
| Variable | HR (95% CI) | P-value |
| Gender (Male vs. Female) | 0.983 (0.398-2.432) | 0.971 |
| Age | 0.995 (0.968-1.022) | 0.695 |
| Stage (Ta vs. T1) | 1.178 (0.568-2.442) | 0.660 |
| Grade (Low vs. High) | 1.012 (0.562-1.824) | 0.967 |
| Tumor size (≦3 cm vs. >3 cm) | 1.006 (0.363-2.792) | 0.990 |
| Number of tumors (single vs. multiple) | 0.871 (0.339-2.242) | 0.775 |
| Intravesical therapy (No vs. Yes) | 0.331 (0.135-0.809) | 0.015 |
| CCNB1 signature (LC vs. HC*) | 2.930 (1.302-6.594) | 0.009 |

*Outcome from the unsupervised hierarchical clustering in FIG. 1 was used for the analysis (low CCNB1 cluster [LC] or high CCNB1 cluster [HC]).
Abbreviations:
HR, hazard ratio;
CI, confidence interval

### Example 4

### Determination of Treatment Plan of Non-Muscle Invasive Bladder Cancer Patient after Surgery Depending on Expression Level of CCNB1 Gene

In the case of a non-muscle invasive cancer patient, after transurethral resection (TUR), an intravesical therapy (IVT) is mainly carried out.

The IVT is carried out with Bacillus Calmette-Guerin (BCG) and mitomycin C. Although there are many different views on effects thereof, the IVT has been carried out in many urological hospitals. Recurrence rates depending on an expression level of CCNB1 gene as a recurrence gene of the present disclosure were compared between a patient undergoing the IVT and a patient without undergoing the IVT.

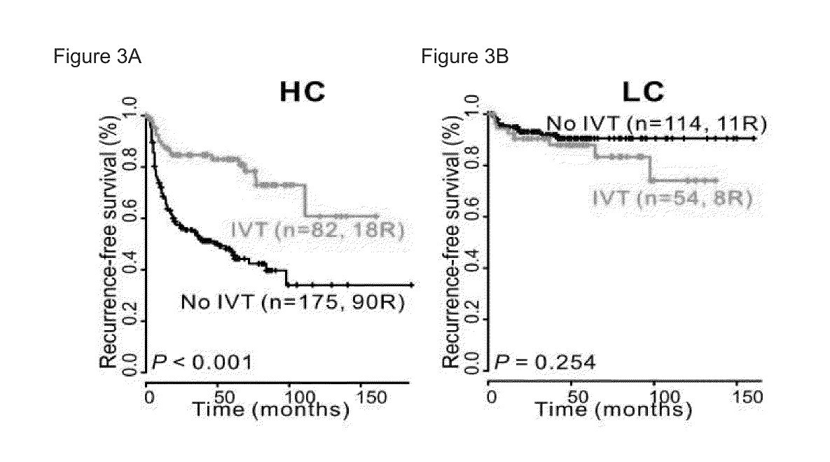

As a result thereof, there was no effect caused by the IVT in the group with a low expression level of CCNB1 (LC: Low Expression of CCNB1). However, it was observed that in the group with a high expression level of CCNB1 (HC: High Expression of CCNB1), recurrence cases were remarkably reduced due to the IVT (refer to FIG. 3).

The above result means that even if non-muscle invasive cancer patients are in the same stage, they can receive different treatments after tumor removal, and is expected to be a helpful indicator at the time of determining a drug treatment plan after non-muscle invasive cancer surgery by performing the IVT to only the group with a high expression level of CCNB1 (HC: High Expression of CCNB1) among non-muscle invasive cancer patients undergoing surgery.

### Example 5

### CCNB1 Gene Expression-Related Gene Network Analysis

In order to find relevance between expression of CCNB1 noted in the present disclosure and recurrence through interactions with other genes, the following experiment was carried out.

Firstly, the inventors of the present disclosure inputted expression difference values of 1393 genes, of which expression patterns are similar to an expression pattern of CCNB1 between the group with HC and the group with LC, searched bibliographic database information relevant to interactions among the 1393 genes, and selected gene groups of which interactions were reported the most among the 1393 genes. Gene interaction bibliographic database search and calculation of rank were carried out using the commercial software Ingenuity Pathway Analysis, and the manufacturer continuously update documents relevant to interactions of genes and also provides a scoring method for calculation of rank.

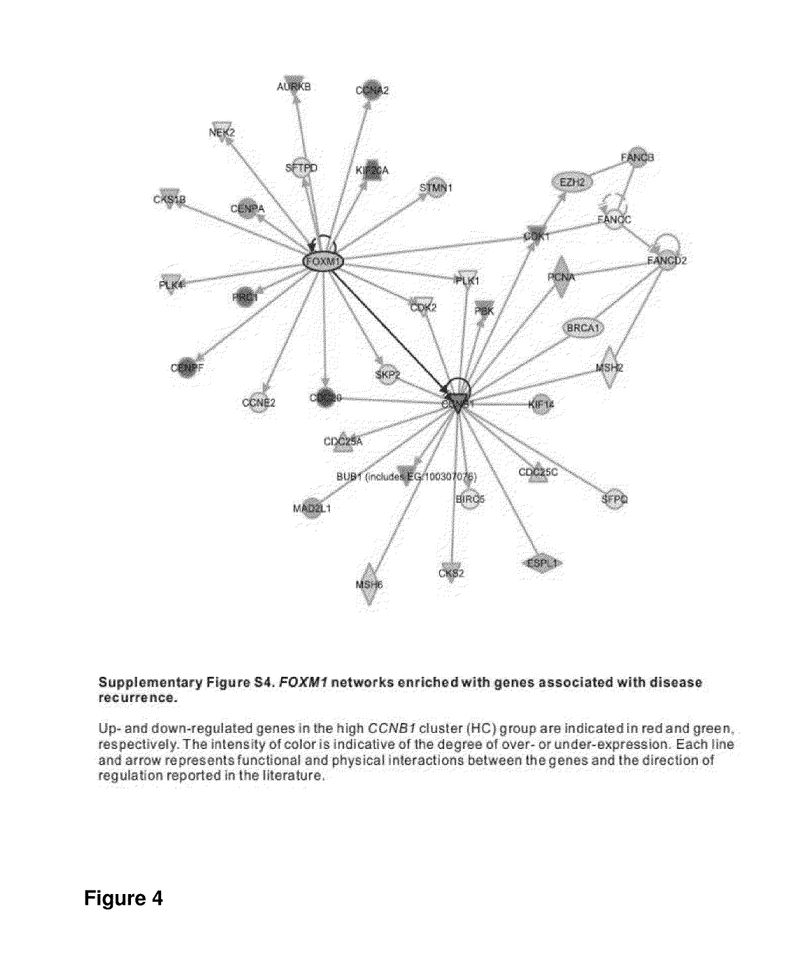

As a result of search for bibliographic database information relevant to interactions among the 1393 genes, it was found that a considerable number of genes (147 genes) relevant to DNA-repair and replication are contained in the 1393 genes, and as a result of interactions among the genes relevant to DNA-repair and replication, a gene network related to FOXM1 and CCNB1 genes as in FIG. 4, together with genes related to Fanconi anemia pathways was obtained.

As a result, it was found that recurrence was significantly increased in the patients with high expression of FANCB, FANCC, and FANCD2 genes related to Fanconi anemia pathways together with the CCNB1 and FOXM1 genes (refer to FIG. 5).

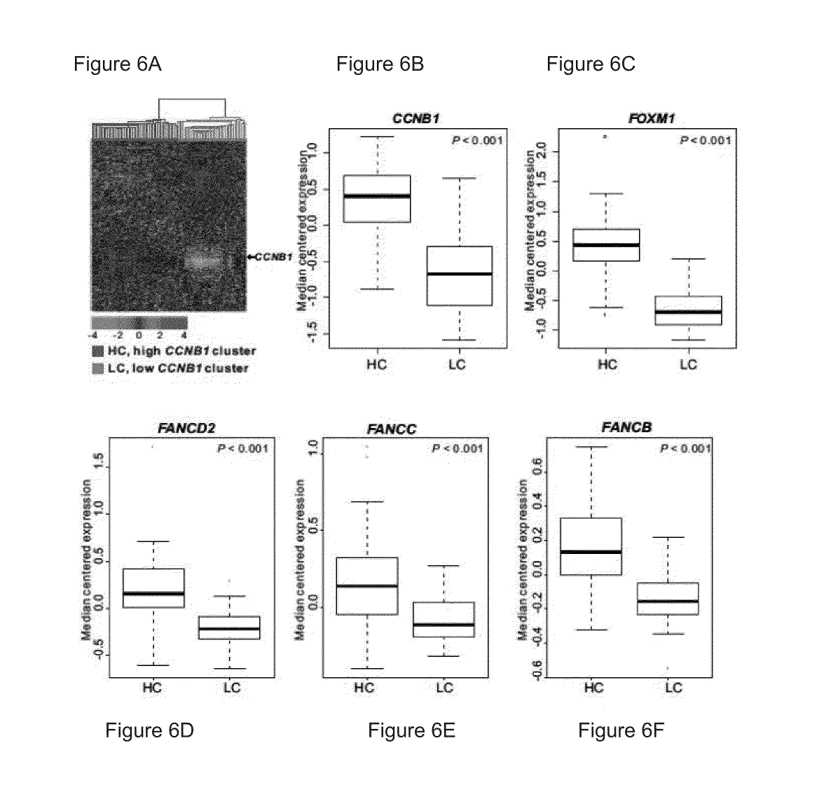

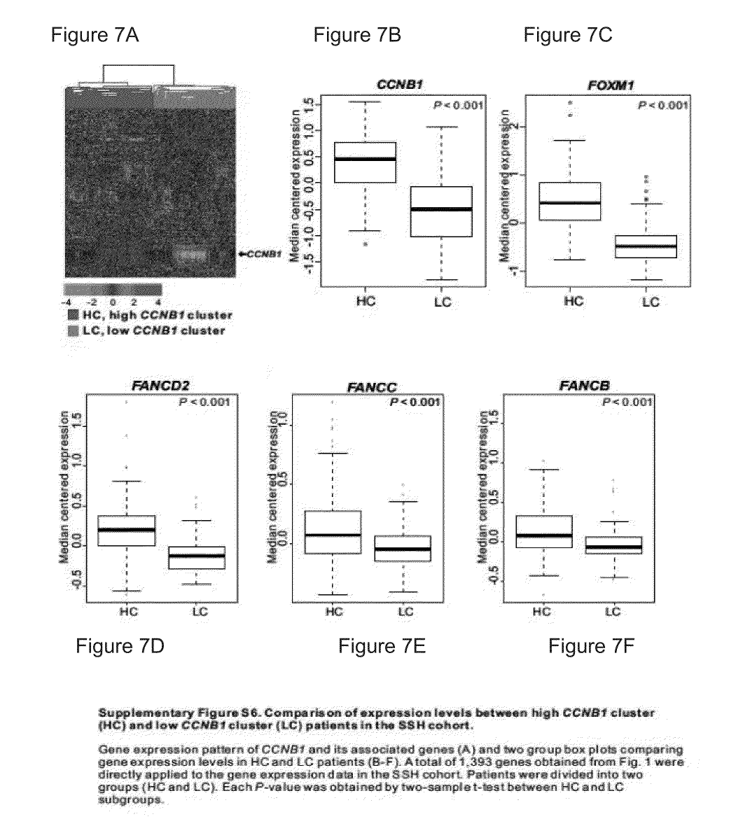

Further, such a result showed that in the other bladder cancer patient group of the Swedish patient group and the Skåne University Hospital patient group in a public domain, a significant result was obtained (refer to FIG. 6 and FIG. 7).

Accordingly, in the patients undergoing surgery for non-muscle invasive bladder cancer, high expression of the above-described five genes results in high recurrence, and, thus, they are required to be treated differentially from general non-muscle invasive cancer patients. The CCNB1, FOXM1, FANCB, FANCC, and FANCD2 genes are suitable to be used as biomarkers for predicting recurrence of non-muscle invasive bladder cancer.

### Example 6

### Check of Gene Expression in Primary Tumor Tissue and Recurrent Tumor Tissue

The inventors of the present disclosure conducted the following experiment in order to check expression of FOXM1 and CCNB1 genes in primary tumor tissues and recurrent tumor tissues.

Microarray is carried out by extracting RNA to synthesize cDNA and then synthesizing the synthesized cDNA to cRNA.

Firstly, cDNA was synthesized with 500 ng of RNA extracted from the tissue and cell line of a patient. For synthesis of cRNA, an (Ambion) Illumina TotalPrep RNA Amplification Kit manufactured by Illumina was used. In a first process of synthesis, RNA, T7 oligo (dT) primer, 10× first strand buffer, dNTP mix, RNase inhibitor, and array script were mixed and reacted at 42° C. for 2 hours. In a second process, the primary product produced in the first process was mixed and reacted with nuclease-free water, 10× second strand buffer, dNTP, DNA polymerase, and RNase H at 16° C. for 2 hours.

The completed cDNA was purified, and then, cRNA synthesis was carried out. In the cRNA synthesis process, the cDNA was mixed and reacted with T7 10× reaction buffer, T7 enzyme mix, and biotin-NTP mix at 37° C. for 14 hours. The completed cRNA was purified, and a beadchip (HumanHT_{—}12_v4 BeadChip For Gene Expression) was used in a hybridization process.

In the hybridization process, 750 ng of the cRNA was mixed and reacted with GEX-HYB buffer at 65° C. for 5 minutes and left at room temperature for 2 minutes, and the mixture was divided in the beadchip. After division, the GEX-HYB buffer was divided in a chamber, and the beadchip was put into the chamber to make a reaction at 58° C. for 16 to 20 hours. After the reaction was completed, the beadchip was washed and then scanned.

A real-time PCR was carried out by synthesizing cDNA with 2 μg of RNA extracted from a cell line of a patient and mixing 50 ng of the synthesized cDNA with SYBR Green dye and a primer (10 pmole).

In this case, temperature conditions included 95° C. for 5 minutes during a denaturation process, 95° C. for 10 seconds during an annealing process, and 60° C. for 30 seconds during an extension process.

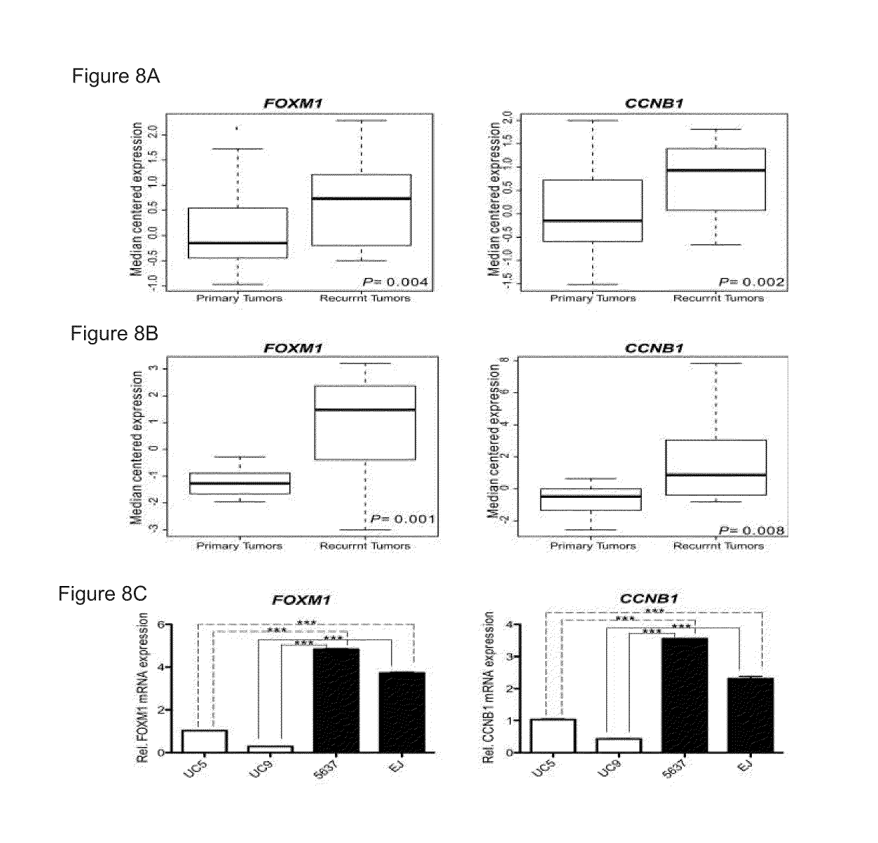

As a result of the microarray and the real-time PCR, it could be seen that slight expression of the two genes was shown in the primary tumor tissues, whereas high expression was shown in the recurrent tumor tissues (refer to FIGS. 8A and 8B). The above result means that cancer recurrence is highly relevant to expression of the FOXM1 and CCNB1 genes.

In addition, in order to carry out the experiment using a bladder cancer cell line, expression levels of the FOXM1 and CCNB1 genes in non-muscle invasive cell lines (UC5, UC9) and invasive cell lines (5637, EJ) were checked, and as a result, it was confirmed that expression of the FOXM1 and CCNB1 genes in the invasive cell lines was also high (refer to FIG. 8C).

### Example 7

### Check of Gene Expression in Bladder Cancer Cell Line Treated with Anticancer Drug

Further, the inventors of the present disclosure studied expression of FOXM1, CCNB1 genes, and FANCB, FANCC, and FANCD2 genes relevant to Fanconi anemia pathways after non-muscle invasive cell lines (UC5, UC9) and invasive cell lines (5637, EJ) were treated with doxorubicin used as an anticancer drug for bladder cancer at a concentration of 0 to 50 μM for 12 hours or 24 hours.

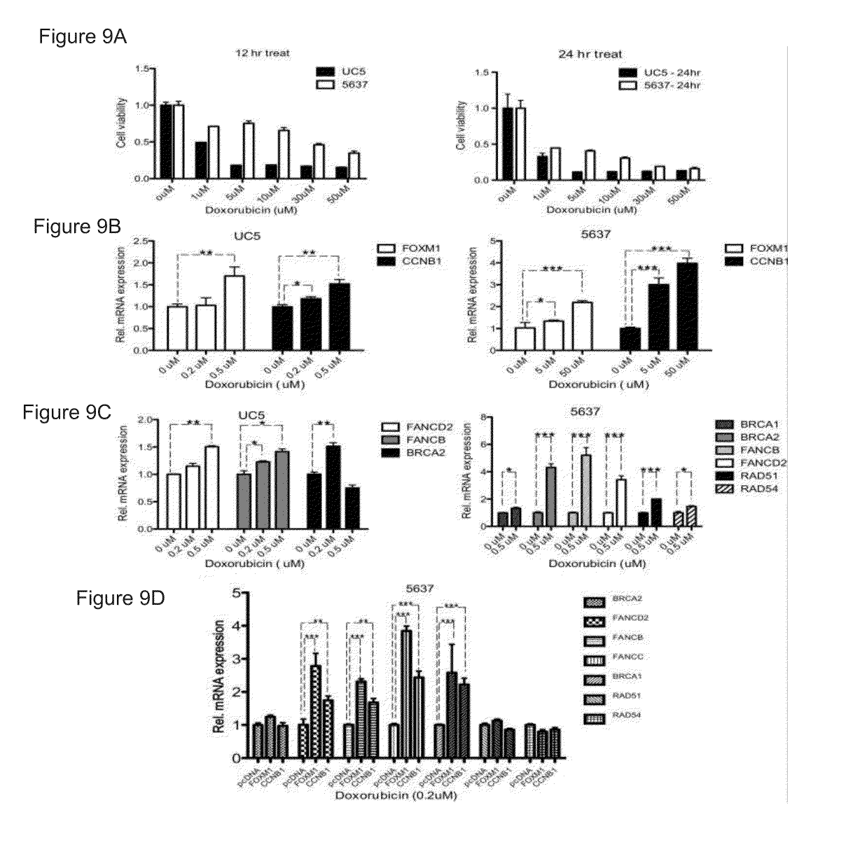

As a result thereof, it was found that a cell viability was high in the invasive cell lines (5637, EJ) (refer to FIG. 9A), and expression of FOXM1 and CCNB1 genes was also high (refer to FIG. 9B).

Furthermore, in the non-muscle invasive cell lines (UC5, UC9) and invasive cell lines (5637, EJ) treated as described above, expression of the genes relevant to DNA repair was checked. As a result thereof, it was found that expression of the FANCB, FANCC, and FANCD2 genes relevant to Fanconi anemia pathways was high (refer to FIG. 9C).

The inventors of the present disclosure studied expression of the FANCB, FANCC, and FANCD2 genes relevant to Fanconi anemia pathways in the 5637 cell line where the FOXM1 and CCNB1 genes were overexpressed in order to check whether expression patterns of such genes are directly regulated by the FOXM1 and CCNB1 genes. As a result thereof, it was found that overexpression of the FOXM1 and CCNB1 genes increased expression of the subordinate FANCB, FANCC, and FANCD2 genes (refer to FIG. 9D).

Accordingly, it was found that the CCNB1, FOXM1, FANCB, FANCC, and FANCD2 genes of the present disclosure are suitable to be used as biomarkers for predicting recurrence of non-muscle invasive bladder cancer, and information for determining a treatment plan of a non-muscle invasive bladder cancer patient after surgical tumor removal can be provided using such a result.

While the present disclosure has been shown and described with reference to preferable Examples thereof, it will be understood by those skilled in the art that various changes in form and details may be made therein without departing from the spirit and scope of the present disclosure as defined by the appended claims. Therefore, the disclosed Examples should not be considered in view of explanation, but no limitation. The technical scope of the present disclosure is taught in the claims, but not the detailed description and all the differences in the equivalent scope thereof should be construed as falling within the present disclosure.

From the foregoing, it will be appreciated that various embodiments of the present disclosure have been described herein for purposes of illustration, and that various modifications may be made without departing from the scope and spirit of the present disclosure. Accordingly, the various embodiments disclosed herein are not intended to be limiting, with the true scope and spirit being indicated by the following claims.

What is claimed is:

1. A method of diagnosing a possibility of bladder cancer recurrence, the method comprising:
a step of measuring an expression level of at least one gene selected from the group consisting of CCNB1, FOXM1, FANCB, FANCC, and FANCD2.

2. The method of diagnosing a possibility of bladder cancer recurrence of claim 1, further comprising:
a step of comparing an expression level of at least one gene selected from the group consisting of CCNB1, FOXM1, FANCB, FANCC, and FANCD2 with respect to a specimen obtained from a subject from which non-muscle invasive bladder cancer is removed with an expression level of the gene expressed in a specimen obtained from a normal subject without bladder cancer.

3. The method of diagnosing a possibility of bladder cancer recurrence of claim 1,
wherein the measurement is carried out by measuring a level of mRNA of the gene or protein

4. The method of diagnosing a possibility of bladder cancer recurrence of claim 2,
wherein when the expression level of at least one gene selected from the group consisting of CCNB1, FOXM1, FANCB, FANCC, and FANCD2 is higher than the expression level expressed in the normal subject, a degree of risk of bladder cancer recurrence is determined as being high.

5. A method of providing information of a personalized medicine after surgical tumor removal, the method comprising:
a step of measuring an expression level of at least one gene selected from the group consisting of CCNB1, FOXM1, FANCB, FANCC, and FANCD2.

6. The method of providing information of a personalized medicine after surgical tumor removal of claim 5,
wherein when the expression level of the gene is higher than an expression level of the gene expressed in a specimen obtained from a normal subject without bladder cancer, it is determined to carry out a treatment with Bacillus Calmette-Guerin (BCG) or mytomycin C.

7. A method of predicting a possibility of bladder cancer recurrence, the method comprising:
a step of measuring an expression level of at least one gene selected from the group consisting of CCNB1, FOXM1, FANCB, FANCC, and FANCD2.

8. The method of predicting a possibility of bladder cancer recurrence of claim 7, further comprising:
a step of comparing an expression level of at least one gene selected from the group consisting of CCNB1, FOXM1, FANCB, FANCC, and FANCD2 with respect to a specimen obtained from a subject from which non-muscle invasive bladder cancer is removed with an expression level of the gene expressed in a specimen obtained from a normal subject without bladder cancer.

9. The method of predicting a possibility of bladder cancer recurrence of claim 7,
wherein the measurement is carried out by measuring a level of mRNA of the gene or protein.

10. The method of predicting a possibility of bladder cancer recurrence of claim 8,
wherein when the expression level of at least one gene selected from the group consisting of CCNB1, FOXM1, FANCB, FANCC, and FANCD2 is higher than the expression level expressed in the normal subject, a degree of risk of bladder cancer recurrence is determined as being high.
